(19) United States
(12) Patent Application Publication
(10) Pub. No.: US 2017/0175043 A1
(43) Pub. Date: Jun. 22, 2017

(54) Detergent Composition, Method and Use of Detergent Composition

(71) Applicant: Novozymes A/S, Bagsvaerd (DK)

(72) Inventors: Peter Dybdahl Hede, Valby (DK); Jan Peter Skagerlind, Hellsinborg (SE)

(73) Assignee: Novozymes A/S, Bagsvaerd (DK)

(21) Appl. No.: 15/116,854

(22) PCT Filed: Feb. 4, 2015
(86) PCT No.: PCT/EP2015/052336

§ 371 Date: Aug. 5, 2016

(30) Foreign Application Priority Data
Feb. 11, 2014 (EP) 14154631.7

(51) Int. Cl. C11D 3/386; C12N 9/26; C12N 9/48; C12N 9/20; C12N 9/24; C12N 9/88; C11D 11/00; C12N 9/42

(52) U.S. Cl. CPC C11D 3/386; C11D 3/38627; C11D 3/38636; C11D 3/38645; C11D 11/0017; C12N 9/2414; C12N 9/2437; C12N 9/20; C12N 9/2491; C12N 9/88; C12N 9/48; C12Y 302/01001; C12Y 302/01004; C12Y 301/01003; C12Y 302/01078; C12Y 402/02002; C12Y 304/00

(57) ABSTRACT

The present invention concerns a detergent composition comprising a surfactant and a multi enzyme composition comprising at least three enzymes selected from the group consisting of: protease, amylase, lipase, mannanase, pectate lyase and cellulase. Further disclosed is the use of the detergent composition and methods, wherein the compositions are used.

### REFERENCE TO A SEQUENCE LISTING

This application contains a Sequence Listing in computer readable form. The computer readable form is incorporated herein by reference.

### FIELD OF THE INVENTION

The present invention concerns a multi enzyme composition, a detergent composition comprising a multi enzyme composition, use of the multi enzyme composition and methods wherein the multi enzyme compositions are used.

### BACKGROUND OF THE INVENTION

Household cleaning is a daily worldwide activity, which means that billions of people use detergent to clean their clothes, dishes and so forth. Laundering is in general performed by agitating the fabrics in a detergent solution for a certain period of time followed by rinsing the fabrics in water. Laundering may be carried out by hand or by using an automatic washing machine in domestic household or in industrial scale.

Much progress has been made in the last decades in sawing energy during cleaning processes e.g. by lowering the temperature of the wash liquor in laundry processes.

However, there is still a need for new wash processes with reduced energy consumption.

### SUMMARY OF THE INVENTION

The present invention concerns a multi enzyme composition comprising at least three enzymes selected from the group consisting of: protease, amylase, lipase, mannanase, pectate lyase and cellulase.

The invention further concerns a detergent composition comprising the multi enzyme composition.

Additionally, the invention concerns the use of a multi enzyme composition for reducing the time period of a wash cycle for washing a soiled item, the use of a multi enzyme composition for maintaining or improving whiteness, the use of a multi enzyme composition for reducing redeposition of soil and the use of a multi enzyme composition for removing or releasing the constituent of a skin-derived body soil deposition from a textile, e.g. by reduction of yellowing.

The invention also concerns a method for reducing the time period of a wash cycle, a method for maintaining or improving the whiteness, a method for reducing redepostion of soil, a method for removing or releasing the constituents of a skin-derived body soil deposition from a textile having a skin-derived body soil deposition. The invention further concerns an Item treated according to the inventive methods.

### BRIEF DESCRIPTION OF THE FIGURES

FIG. 1: The wash performance on Carrot baby food stain (WE5IACBFWKC) of the three formulations after varying washing times.

FIG. 2: The wash performance on Rice starch (CS-28) of the three formulations after varying washing times.

FIG. 3: The wash performance on Fresh Banana (Equest 013A KC) of the three formulations after varying washing times and at 20° C. and 40° C.

FIG. 4: The sum of wash performance (measured as the sum of the four used whiteness tracers at 460 nm Remission) on four different well-known whiteness tracers being WFK 10A, WFK 20A, WFK 30A and EMPA 221 of the three formulations after varying washing times at 20° C. and 40° C.

FIG. 5: A picture of a collar cut in two equal pieces in the middle of the collar.

### DEFINITIONS

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

“Tyr167Gly+Arg170Gly”, “Tyr167Gly+Arg170Ala”, “Tyr167Ala+Arg170Gly”, and “Tyr167Ala+Arg170Ala”.

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Benchmark: The terms “Benchmark” or “Benchmark washing” or “Benchmark washing conditions” in relation to a method of the invention are defined herein as completion of a washing cycle with a detergent composition without enzymes (benchmark detergent composition), wherein the washing cycle is carried out for 120 minutes.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Delta enzyme performance value: The term “Delta enzyme remission value” or “delta Enz” or “ΔEnz” is defined herein as the result of a reflectance or remission measurement at 460 nm. The swatch is measured with one swatch of similar color as background, preferably a swatch from a repetition wash. A swatch representing each swatch type is measured before wash. The delta enzyme performance value is the remission value of the swatch washed in detergent with an enzyme present minus the remission value of a similar swatch washed in a detergent without enzyme present.

Delta remission value (ΔRem): The terms “Delta remission” or “Delta remission value” are defined herein as the result of a reflectance or remission measurement at a certain wavelength which typically is 460 nm. The swatch is measured with one swatch of similar colour as background, preferably a swatch from a repetition wash. A swatch representing each swatch type is measured before the wash. The Delta remission is the remission value of the washed swatch minus the remission value of the unwashed swatch.

Detergent Composition: The term “detergent composition” refers to compositions that find use in the removal of undesired compounds from items to be cleaned, such as textiles, dishes, and hard surfaces. The detergent composition may be used to e.g. clean textiles, dishes and hard surfaces for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish wash detergents). In addition to containing a bifunctional compound of the invention, the detergent formulation may contain one or more additional enzymes (such as proteases, amylases, lipases, cutinases, cellulases, endoglucanases, xyloglucanases, pectinases, pectin lyases, xanthanases, peroxidaes, haloperoxygenases, catalases and mannanases, or any mixture thereof), and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Dish wash: The term “dish wash” refers to all forms of washing dishes, e.g. by hand or automatic dish wash (ADW). Washing dishes includes, but is not limited to, the cleaning of all forms of crockery such as plates, cups, glasses, bowls, all forms of cutlery such as spoons, knives, forks and serving utensils as well as ceramics, plastics, metals, china, glass and acrylics.

The term “enzyme variant” means an enzyme comprising differences in its amino acid sequence from that of the parent enzyme. The differences comprise substitutions, deletions and/or insertions as compared to the parent enzyme.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.

Improved wash performance: The term “improved wash performance” is defined herein as detergent comprising the multi enzyme composition displaying an increased wash performance relative to the wash performance of the detergent composition without enzymes e.g. by increased stain removal. The term “improved wash performance” includes wash performance in laundry but also e.g. in hard surface cleaning such as automated dish wash (ADW).

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases, the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

The term “parent enzyme” as used herein means an enzyme in which modifications are being made to produce the enzyme variants of the invention. A parent enzyme may be an enzyme isolated from a natural source, or an enzyme wherein previous modification(s) have been made while retaining the characteristic activity of the enzyme in question. The parent pectate lyase of the invention may be a wild-type pectate lyase.

Multi enzyme composition: The term “multi-enzyme composition” is defined herein to mean a composition comprising protease, amylase, lipase, mannanase, pectate lyase, and cellulase. having a wash effect in laundry. Examples of wash effects in laundry can be for example enzymes that are known to remove certain stains, enzymes that are known to have a anti re-deposition effect (also known as anti-greying) or enzymes that remove yellowing in laundry.

Remission value: The terms “Remission” or “Remission value” are defined herein as the result of a reflectance or remission measurement at a certain wavelength which typically is 460 nm.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well.

Wash cycle: The term “wash cycle” is defined herein as a washing operation wherein textiles are immersed in the wash liquor, mechanical action of some kind is applied to the textile in order to release stains and to facilitate flow of wash liquor in and out of the textile and finally the superfluous wash liquor is removed. After one or more wash cycles, the textile is generally rinsed and dried.

Wash liquor: The term “wash liquor” is defined herein as the solution or mixture of water and detergents optionally including the multi enzyme composition used for laundering textiles.

Wash time: The term “wash time” is defined herein as the time it takes for the entire washing process; i.e. the time for the wash cycle(s) and rinse cycle(s) together.

Whiteness: The term “Whiteness” is defined herein as a broad term with different meanings in different regions and for different consumers. Loss of whiteness can e.g. be due to greying, yellowing, or removal of optical brighteners/hueing agents. Greying and yellowing can be due to soil redeposition, body soils, colouring from e.g. iron and copper ions or dye transfer. Whiteness might include one or several issues from the list below: colourant or dye effects; incomplete stain removal (e.g. body soils, sebum etc.); redeposition (greying, yellowing or other discolourations of the object) (removed soils reassociate with other parts of textile, soiled or unsoiled); chemical changes in textile during application; and clarification or brightening of colours.

### DETAILED DESCRIPTION OF THE INVENTION

The present invention concerns a multi enzyme composition comprising at least the following enzymes: protease, amylase, lipase, mannanase, pectate lyase and cellulase. The present invention further concerns a detergent composition comprising a surfactant and a multi enzyme composition comprising at least the following enzymes: protease, amylase, lipase, mannanase, pectate lyase and cellulase.

The invention further concerns the use of a multi enzyme composition for reducing the time period of a wash cycle for washing a soiled item, wherein the multi enzyme composition comprises at least the following enzymes: protease, amylase, lipase, mannanase, pectate lyase and cellulase.

The invention further concerns the use of a multi enzyme composition for maintaining or improving whiteness, wherein the multi enzyme composition comprises at least the following enzymes: protease, amylase, lipase, mannanase, pectate lyase and cellulase.

The invention further concerns the use of a multi enzyme composition for reducing redeposition of soil, wherein the multi enzyme composition comprises at least the following enzymes: protease, amylase, lipase, mannanase, pectate lyase and cellulase.

The invention further concerns the use of a multi enzyme composition for removing or releasing the constituents of a skin-derived body soil deposition from a textile having a skin-derived body soil deposition, wherein the multi enzyme composition comprises at least the following enzymes: protease, amylase, lipase, mannanase, pectate lyase and cellulase.

The invention also concerns a method for reducing the time period of a wash cycle for washing a soiled item, wherein the item is exposed to wash liquor comprising an multi enzyme composition and a wash cycle is completed, wherein said multi enzyme composition comprises at least the following enzymes: protease, amylase, lipase, mannanase, pectate lyase and cellulase.

Further, the invention concerns a method for maintaining or improving the whiteness wherein a textile is exposed to wash liquor comprising a multi enzyme composition, wherein said multi enzyme composition comprises at least the following enzymes: protease, amylase, lipase, mannanase, pectate lyase and cellulase.

The invention further concerns a method for removing or releasing the constituents of a skin-derived body soil deposition from a textile having a skin-derived body soil deposition, wherein the multi enzyme composition comprises at least the following enzymes: protease, amylase, lipase, mannanase, pectate lyase and cellulase.

In the uses and methods described above, the multi enzyme composition can be comprised in a detergent composition comprising a surfactant.

The invention further concerns an item treated according to the inventive method.

The inventors of the present invention have found that by using a multi enzyme composition in a laundry process it is possible to reduce the amount of time used for washing. This is of advantage to the environment because less energy is used. Another advantage is that the consumer saves time for washing. This is off course of great importance in domestic household but even more important in industrial washing processes, where time is of even greater importance.

Furthermore, it is surprisingly found that even though the time for washing is reduced the whiteness of the textile is maintained or improved by the use of the multi enzyme composition according to the invention. Results have shown that when using a multi enzyme composition it is possible to both reduce the amount of surfactant in a formulation to almost half the level while at the same time maintaining or even improving the performance.

The amount of surfactant may be reduced so that the amount of surfactant is below 40% w/w of detergent composition, below 35% w/w of detergent composition, below 30% w/w of detergent composition, below 25% w/w of detergent composition or below 20% w/w of detergent composition.

The multi enzyme composition of the invention comprises at least the following enzymes: protease, amylase, lipase, mannanase, pectate lyase and cellulase.

The protease may be of animal, vegetable or microbial origin. In one embodiment of the invention the protease is chemically modified or protein engineered.

In one embodiment, the protease is a serine protease or a metalloprotease, preferably an alkaline microbial protease or a trypsin-like protease. In one embodiment the protease is selected from the group consisting of Bacillus, e.g., subtilisin Novo, subtilisin Carlsberg, subtilisin 309, subtilisin 147, subtilisin 168, trypsin of bovine origin, trypsin of porcine origin and Fusarium protease.

In one embodiment, the protease is an alkaline protease having at least 90% identity to the amino acid sequence of SEQ ID NO: 1 or a variant thereof with substitutions in one or more of the following positions: 27, 36, 57, 76, 87, 97, 101, 104, 120, 123, 167, 170, 194, 206, 218, 222, 224, 235, and 274, preferably the variant is an alkaline protease having at least 90% identity to the amino acid sequence of SEQ ID NO: 1 with the following substitution: M222S.

The multi enzyme composition of the invention may further comprise an amylase. The amylase is an alpha-amylase or a glucoamylase, which is of bacterial or fungal origin. For example the amylase can be an alpha-amylase obtained from Bacillus, such as Bacillus licheniformis.

In one embodiment, the amylase is an alpha-amylase having SEQ ID NO: 2 or a variant thereof having at least 80%, at least 85% or at least 90% sequence identity to SEQ ID NO: 2 and having a substitution, a deletion or an insertion of one amino acids downstream for the amino acid corresponding to the positions in the amylase having SEQ ID NO: 2: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484; Particular preferred amylases include such a variant having a deletion of D183 and G184 and having the substitutions R118K, N195F, R320K and R458K and a variant additionally having substitutions in one or more positions selected from the group: M9, G149, G182, G186, M202, T257, Y295, N299, M323, A339 and E345, most preferred a variant additionally having substitutions in all these positions; or a variant alpha-amylase derived from a parent α-amylase derived from B. licheniformis comprising the mutation:

A1*+N2*+L3V+M15T+R23K+S29A+A30E+Y31H+A33S+E34D+H35I+M197T

In one embodiment of the invention, the multi enzyme composition further comprises a lipase. The lipase is of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™, Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the 554V variant used in the commercial product Gentle Power Bleach from Huntsman Textile Effects Pte Ltd (WO10/100028).

In one embodiment of the invention, the lipase is a polypeptide having the amino acid sequence which:
a. has at least 90% identity with the wild-type lipase derived from Humicola lanuginosa strain DSM 4109; compared to said wild-type lipase,
b. comprises a substitution of an electrically neutral or negatively charged amino acid at the surface of the three-dimensional structure within 15 Å of E1 or Q249 with a positively charged amino acid; and
c. comprises a peptide addition at the C-terminal; and/or
d. meets the following limitations:
   a. comprises a negative amino acid in position E210 of said wild-type lipase;
   b. comprises a negatively charged amino acid in the region corresponding to positions 90-101 of said wild-type lipase; and
   c. comprises a neutral or negative amino acid at a position corresponding to N94 of said wild-type lipase and/or has a negative or neutral net electric charge in the region corresponding to positions 90-101 of said wild-type lipase.

In one embodiment, the substitution mentioned under b) is within 10 Å of E1 or Q249. In one embodiment said substitution is within 15 Å (preferably 10 Å) of E1. In one embodiment, the lipase comprises 2-4 of said substitutions, where 2 substitutions are preferred. In one embodiment, the substitution is of an electrically neutral or negatively charged amino acid within 10 Å of E1 with R. In one embodiment, a substitution of amino acid S3, S224, P229, T231, N233, D234 or T244 with a positively charged amino acid, preferably R.

In one embodiment, 1-5 amino acids have been added to the C-terminal of the peptide.

In one embodiment, the amino acids consist of electrically neutral (preferably hydrophobic) amino acids and are most preferably PGL or PG.

In one embodiment of the invention, the amino acids added at the C-terminal consist of neutral (preferably hydrophobic) amino acids and C, and the lipase further comprises substitution of an amino acid with C so as to form a disulfide bridge with the C of the peptide addition.

In one embodiment, the lipase comprises amino acids with negative or unchanged electric charge in at least two of positions N94, D96 and E99 of said wild-type lipase.

In one embodiment, the lipase comprises a substitution G91A, N94D/E/K/R, D96E/I/L/N/S/W, E99N/Q/K/R/H, N101S, R209P/S or Q249R/K/H.

In one embodiment of the invention, the lipase comprises one of the following sets of substitutions, optionally combined with Q249R/K/H and/or K98X:

G91G/A/S/T+N94 (neutral or negative)+D96D/E/C/Y+E99E/D/C/Y,

G91G/A/S/T+N94 (neutral or negative)+D96D/E/C/Y+E99N+N101S,

G91G/A/S/T+N94R/K/H+D96D/E/C/Y+E99E/D/C/Y,

G91G/A/S/T+N94 (neutral or negative)+D96 (neutral or positive)+E99E/D/C/Y, or

G91G/A/S/T+N94 (neutral or negative)+D96D/E/C/Y+E99 (neutral or positive).

In one embodiment, the lipase comprises one of the following sets of substitutions, optionally combined with R209 (neutral or negative):

E99R/K/H+Q249R/K/H,

N94R/K/H+Q249R/K/H, or

D96 (neutral or positive)+Q249R/K/H.

In one embodiment of the invention, the mannanase of the multi enzyme composition has a sequence that is at least 80%, at least 85% or at least 90% identical to SEQ ID NO: 3

In one embodiment of the invention, the multi enzyme composition comprises at least one pectate lyase. The pectate lyase is a variant of a parent enzyme having pectate lyase activity (EC 4.2.2.2) and comprising an alteration at one or more positions selected from the group consisting of positions number: 5, 9, 11, 26, 28, 30, 31, 37, 40, 45, 46, 47, 48, 49, 50, 51, 52, 54, 61, 64, 68, 69, 70, 71, 74, 75, 76, 79, 86, 87, 91, 99, 105, 106, 107, 111, 115, 116, 118, 122, 123, 134, 136, 139, 140, 141, 146, 148, 156, 158, 170, 182, 185, 186, 189, 193, 194, 196, 199, 201, 202, 204, 213, 215, 218, 224, 228, 229, 234, 235, 237, 251, 256, 257, 258, 272, 277, 286, 295, 298, 301, 302, 303, 305, 307, 308, 314, 316, 323, 324, 326, 331, 332, 333, 334, 335, 336, 337, 338, 339, 340, 341, 349, 356, 357, 363, 366, 378, 381, 384, 386, 387, 389, 390, 391, 393 and 397, wherein the alteration(s) are independently:
a. an insertion of an amino acid downstream of the amino acid which occupies the position,
b. a deletion of the amino acid which occupies the position, or
c. a substitution of the amino acid which occupies the position with a different amino acid, and

wherein each position corresponds to a position of the amino acid sequence of the pectate lyase having the amino acid sequence of SEQ ID NO:4, and wherein the parent enzyme is the pectate lyase shown on SEQ ID NO:4 or a pectate lyase having at least 65% identity to the amino acid sequence of SEQ ID NO:4.

The variant has improved detergent stability compared to the parent enzyme.

In one embodiment of the invention, the alteration or alterations are one or more substitutions.

In one embodiment of the invention, the one or more substitutions in the pectate lyase variant are selected from the group consisting of: H5R, E9G, N11Y, K26Q, S28T, S30F, 530P, S30T, H31N, N37D, Q40E, L45V, G46D, K47N, K47R, D48E, D48P, T49P, N50D, N50L, N50Y, N51Y, T52M, K54V, T61A, M64F, D68*, N69*, L70*, K71*, K71E, G74D, L75A, L75P, N76D, K79A, D86N, K87A, K87E, A91E, K991, K99N, K99R, T105A, T105P, L106Q, E107K, A111E, K115A, K1151, K115N, K115Q, N116D, K118A, K118E, M122E, M122K, M122N, M122Q, V123I, S134L, T136S, K139E, K139F, K1391, K139M, K139N, K139S, 1140V, V141G, V141E, V141L, V141N, Q146F, Q146H, Q146I, Q146V, K148E, K148Q, N156S, E158N, D170N, Q182D, Q182E, N185H, N186H, N189D, H193Y, I194V, I196V, C199N, C199S, F201L, N202K, G204R, K213E, K213N, K213T, F215Y, K218E, K218L, K218P, G224S, A2281, S229T, Y234H, I235V, M237I, F251I, S256C, K257E, K257N, T258I, L286Y, R272C, R272H, R272Y, V277D, G286A, Y295H, S298N, S301Y, S302A, D303S, A305P, S307R, Y308S, K314N, S316F, N323M, V324A, D326N, S331P, S331T, A332P, A333E, K334E, T335S, T335R, I336S, S337C, S337K, S337L, S337R, V338E, V338Y, F3391, S340A, S340K, S340N, S340P, S340Q, G341S, G349R, Q356H, I357V, N363S, S366N, T378G, T378S, A381D, H384N, K386P, K386R, S387A, V389I, I390N, I390T, S391N, A393V and K397D, and wherein each position corresponds to a position of the amino acid sequence of the pectate lyase having the amino acid sequence of SEQ ID NO:4.

In one embodiment, the parent pectate lyase has an amino acid sequence which has a degree of identity to the amino acid sequence of SEQ ID NO:4 of at least about 70%, preferably at least about 80%, more preferably at least about 90%, even more preferably at least about 95%, and most preferably at least about 97%.

In one embodiment, the parent pectate lyase is encoded by a polynucleotide which hybridizes under medium, more preferably high stringency conditions, with the polynucleotide of SEQ ID NO: 3 or its complementary strand.

The alterations are introduced by random or site-directed mutagenesis or by shuffling one or more DNA sequences encoding pectate lyases having at least 65% identity to SEQ ID NO: 4.

The parent pectate lyase may be a wild-type pectate lyase such as a Bacillus pectate lyase.

In one embodiment, the parent pectate lyase is encoded by the polynucleotide present in the plasmid of the strain Bacillus subtilis DSM 14218. The total number of alterations of the parent pectate lyase is thirteen, preferably twelve, more preferably eleven, even more preferably ten, even more preferably nine, even more preferably eight, even more preferably seven, even more preferably six, even more preferably five, even more preferably four, even more preferably three, even more preferably two, and most preferably one.

In one embodiment of the invention, the cellulase is comprised by the enzyme classification EC 3.2.1.4. The cellulase is of bacterial or fungal origin and can be chemically modified or protein engineered. The cellulase can be derived from the genera Bacillus, Pseudomonas, Humicola, Fusarium, Thielavia or Acremonium. In one embodiment of the invention the cellulase is derived from Humicola insolens, Myceliophthora thermophila, Sporotrichum pulverulentum, Fusarium oxysporum, Trichoderma reesei, Thielavia terrestris, Acremonium sp., Acremonium sp. CBS 478.94, Macrophomina phaseolina CBS 281.96, Crinipellis scabella CBS 280.96, Volutella colletotrichoides or Sordaria fimicola ATCC 52644 or Bacillus SP-KSMS237.

In one embodiment of the invention, the cellulase is an enzyme exhibiting endo-beta-1,4-glucanase activity (EC 3.2.1.4), which is selected from one of:
a) a polypeptide encoded by the DNA sequence of positions 1 to 2322 of SEQ ID NO:5;
b) a polypeptide produced by culturing a cell comprising the sequence of SEQ ID NO:5 under conditions wherein the DNA sequence is expressed; or
c) an endo-beta-1,4-glucanase enzyme having a sequence of at least 97% identity to the amino acid sequence of position 1 to position 773 of SEQ ID NO:6.

In one embodiment, the cellulase exhibiting endo-beta-1,4-glucanase activity (EC 3.2.1.4) has a polypeptide having endo-beta-1,4-glucanase activity that is encoded by a polynucleotide that hybridizes with the nucleotide sequence shown in positions 1-2322 of SEQ ID NO:5 under hybridization conditions comprising 5×SSC at 45° C. and washing conditions comprising 2×SC at 60° C. The cellulase may comprise a polypeptide endogeneous to Bacillus sp., DSM 12648. In one embodiment of the invention the cellulase is active at a pH at least in the range of 4-11, preferably 5.5-10.5.

In one embodiment of the invention, an isolated polynucleotide will hybridize to similar sized regions of SEQ ID NO: 5 or a sequence complementary thereto, under at least medium stringency conditions.

In particular, polynucleotides will hybridize to a denatured double-stranded DNA probe comprising either the full sequence encoding the catalytic domain of the enzyme which sequence is shown in positions 1-2322 of SEQ ID NO: 5 or any probe comprising a subsequence of SEQ ID NO: 5 having a length of at least about 100 base pairs under at least medium stringency conditions, but preferably at high stringency conditions as described in detail below. Suitable experimental conditions for determining hybridization at medium, or high stringency between a nucleotide probe and a homologous DNA or RNA sequence involves presoaking of the filter containing the DNA fragments or RNA to hybridize in 5×SSC (Sodium chloride/Sodium citrate, Sambrook et al. (1989) Molecular cloning: A laboratory manual, Cold Spring Harbor lab., Cold Spring Harbor, N.Y.) for 10 min, and prehybridization of the filter in a solution of 5×SSC, 5×Denhardt's solution (Sambrook et al. 1989), 0.5% SDS and 100 μg/ml of denatured sonicated salmon sperm DNA (Sambrook et al. 1989), followed by hybridization in the same solution containing a concentration of 10 ng/ml of a random-primed (Feinberg, A. P. and Vogelstein, B. (1983) Anal. Biochem. 132:6-13), 32P-dCTP-labeled (specific activity higher than 1×109 cpm/microgram) probe for 12 hours at about 45° C. The filter is then washed twice for 30 minutes in 2×SSC, 0.5% SDS at least 60° C. (medium stringency), still more preferably at least 65° C. (medium/high stringency), even more preferably at least 70° C. (high stringency), and even more preferably at least 75° C. (very high stringency).

Molecules to which the oligonucleotide probe hybridizes under these conditions are detected using an x-ray film.

As previously noted, the isolated polynucleotides of the present invention include DNA and RNA. Methods for isolating DNA and RNA are well known in the art. DNA and RNA encoding genes of interest can be cloned in Gene Banks or DNA libraries by means of methods known in the art.

Polynucleotides encoding polypeptides having endo-glucanase activity of the invention are then identified and isolated by, for example, hybridization or PCR.

The present invention further provides counterpart polypeptides and polynucleotides from different bacterial strains (orthologs or paralogs). Of particular interest are endo-glucanase polypeptides from gram-positive alkalophilic strains, including species of Bacillus.

Species homologues of a polypeptide with endo-glucanase activity of the invention can be cloned using information and compositions provided by the present invention in combination with conventional cloning techniques. For example, a DNA sequence of the present invention can be cloned using chromosomal DNA obtained from a cell type that expresses the protein. Suitable sources of DNA can be identified by probing Northern blots with probes designed from the sequences disclosed herein. A library is then prepared from chromosomal DNA of a positive cell line. A DNA sequence of the invention encoding an polypeptide having endo-glucanase activity can then be isolated by a variety of methods, such as by probing with probes designed from the sequences disclosed in the present specification and claims or with one or more sets of degenerate probes based on the disclosed sequences. A DNA sequence of the invention can also be cloned using the polymerase chain reaction, or PCR (Mullis, U.S. Pat. No. 4,683,202), using primers designed from the sequences disclosed herein. Within an additional method, the DNA library can be used to transform or transfect host cells, and expression of the DNA of interest can be detected with an antibody (monoclonal or polyclonal) raised against the endo-glucanase cloned from B. sp., DSM 12648, expressed and purified as described in Materials and Methods and Examples 1 and 2 of WO 2002/099091, or by an activity test relating to a polypeptide having endo-glucanase activity.

The endo-glucanase encoding part of the DNA sequence shown in SEQ ID NO:5 and/or an analogue DNA sequence of the invention may be cloned from a strain of the bacterial species Bacillus sp., preferably the strain DSM12648, producing the enzyme with endo-glucanase activity, or another or related organism as described herein.

How to use a sequence of the invention to get other related sequences: The disclosed sequence information herein relating to a polynucleotide sequence encoding an endo-beta-1,4-glucanase of the invention can be used as a tool to identify other homologous endo-glucanases. For instance, polymerase chain reaction (PCR) can be used to amplify sequences encoding other homologous endo-glucanases from a variety of microbial sources, in particular of different Bacillus species.

The sequence of amino acids in position 1 to position 773 of SEQ ID NO: 6 is a mature endo-glucanase sequence with a calculated molecular weight of 86 kDa. It is believed that positions 1 to about 340 of SEQ ID NO: 6 are the catalytically active domain of the of the present endo-glucanase enzyme. It is also believed that positions from about 340 to about 540 are the cellulose binding domain of the present endo-glucanase enzyme. The function of the remainder of the sequence, i.e. from about position 540 to position 773 is at present unknown.

The present invention provides an endo-glucanase enzyme comprising (i) the amino acid sequence of position 1 to position 773 of SEQ ID NO: 6, or a fragment thereof that has endo-glucanase activity.

A fragment of position 1 to position 773 of SEQ ID NO: 6 is a polypeptide, which have one or more amino acids deleted from the amino and/or carboxyl terminus of this amino acid sequence. In an embodiment the present invention provides an endo-glucanase enzyme comprising (ii) the amino acid sequence of positions 1 to about 340 of SEQ ID NO: 6, since it is contemplated that such a mono-domain endo-glucanase is also useful in the industrial applications described herein. In another embodiment the present invention provides an endo-glucanase enzyme comprising (iii) the amino acid sequence of positions 1 to from between about 540 and 773 of SEQ ID NO: 6, since it is contemplated that such an endo-glucanase comprising the catalytically active domain and the cellulose binding domain is also useful in the industrial applications described herein. In a preferred embodiment, such fragment is a polypeptide which consists of position 1 to position 663±50 amino acids, preferably 1 to 663±25 amino acids.

In one embodiment of the invention, the ratio between the protease and the other enzymes is 5:1, 3:1 or 2:1.

In one embodiment of the invention, the multi enzyme composition may comprise at least one additional enzyme selected from the group consisting of protease, lipase, cutinase, amylase, carbohydrase, cellulase, pectate lyase, pectinase, mannanase, arabinase, galactanase, and/or xylanase.

The concentration of each of the enzymes in the multi enzyme composition are in range of 0.01-3.0% w/w enzyme per detergent composition, in the range of 0.05-2.5% w/w enzyme per detergent composition, in the range of 0.1-2.1% w/w enzyme per detergent composition, in the range of 0.1-2.2% w/w enzyme per detergent composition, in the range of 0.05-2.1% w/w enzyme per detergent composition, in the range of 0.05-2.0% w/w enzyme per detergent composition. In one embodiment of the invention the lipase and the cellulase is used as a composition in the form of a bar, a homogenous tablet, a tablet having two or more layers, a pouch having one or more compartments, a regular or compact powder, a granule, a paste, a gel, or a regular, compact or concentrated liquid.

In one embodiment, the concentration of the enzymes in the detergent composition is:

a. From 0.5-3.0% protease,

b. From 0.01-1.5% amylase,

c. From 0.01-1.0% lipase,

d. From 0.01-1.0% mannanase,

e. From 0.01-0.9% pectate lyase, and

f. From 0.01-1.0% cellulase

calculated as % w/w enzyme of the detergent composition.

In one embodiment, the concentration of the enzymes in the detergent composition is:

a. From 1.5-2.5% protease,

b. From 0.1-0.9% amylase,

c. From 0.1-0.8% lipase,

d. From 0.1-0.8% mannanase,

e. From 0.1-0.5% pectate lyase, and

f. From 0.1-0.8% cellulase

calculated as % w/w enzyme of the detergent composition.

In one embodiment, the concentration of the enzymes in the detergent composition is:

a. 2% protease,

b. 0.5% amylase,

c. 0.3% lipase,

d. 0.3% mannanase,

e. 0.2% pectate lyase, and

f. 0.3% cellulase

calculated as % w/w enzyme of the detergent composition.

The multi enzyme composition may comprise granules, which granules comprise a core and a protective coating. The coating may be a coating as described in WO2011/134809. If the multi enzyme composition is used in a liquid detergent composition, the composition may be stabilized with the compound described in WO 2009/118375.

In one embodiment of the invention, the multi enzyme composition comprises at least the following enzymes: Protease, amylase, lipase, mannanase, pectate lyase and cellulase and may be used for reducing the time period of a wash cycle for washing a soiled item. The item may be a textile, a dish or a hard surface. The reduction of the time period for completing a wash cycle may be due to the use of the multi enzyme composition of the invention and at the same time a raise in the temperature of the wash liquor. The temperature of the wash liquor is in the range of 5° C. to 95° C., or in the range of 10° C. to 80° C., or in the range of 10° C. to 70° C., or in the range of 10° C. to 60° C., or in the range of 10° C. to 50° C., or in the range of 15° C. to 40° C., or in the range of 20° C. to 30° C.

Many stains need different kinds of cleaning chemistry and process in order to be removed. This gives a dilemma because often many different kinds of soling are gathered in the same wash load, which also caused the problem of redeposition of soil. Detergents for a normal wash are formulated as a compromise in respect to cleaning many different stain types in same process at same time. With the present multi enzyme composition a broad range of stains are cleaned and it has further been found that the whiteness of the item is maintained or improved, the redeposition is reduced and the time period used for completing a wash cycle is reduced. The multi enzyme composition may be used either alone or as a component of a detergent composition. The soiled item may a textile, a dish or a hard surface. The performance with regard to whiteness and redeposition of a detergent composition comprising the multi enzyme composition and a reduced amount of surfactant is the same or even improved compared to a detergent composition without the multi enzyme composition.

In one embodiment, the multi enzyme composition comprises at least the following enzymes: Protease, amylase, lipase, mannanase, pectate lyase and cellulase and is used for removing or releasing the constituents of a skin derived body soil deposition.

In one embodiment, the multi enzyme composition comprises at least the following enzymes: Protease, amylase, lipase, mannanase, pectate lyase and cellulase and is used for removing yellowing.

A primary source of so-called yellowing of fabric items such as bed linen, pillow cases, cuffs and collars of shirt is due to accumulation and aging of body soil components. Here, transfer to the textile matrix of sebum and skin residues are of key importance, see J. Surf. Det. (1998), 3: 407-418; J. Surf. Det. (2001), 4: 35-41; and references therein. Accumulation of oxidation labile compounds may upon aging render yellowish, oxidized and cross-linked structures that are very hard to remove.

In one embodiment, the multi enzyme composition comprises at least the following enzymes: Protease, amylase, lipase, mannanase, pectate lyase and cellulase and is used for reducing redepostion of soil.

The invention further concerns a method for washing a soiled item, wherein the item is exposed to a wash liquor and a wash cycle is completed, wherein said multi enzyme composition comprises at least the following enzymes: protease, amylase, lipase, mannanase, pectate lyase and cellulase. The method results in reduced time period for completing a wash cycle, for maintaining or improving the whiteness, for reducing the redeposition, for removing or releasing the constituents of a skin-derived body soil deposition from a textile having a skin-derived body soil deposition or for reducing yellowing. The multi enzyme composition may be used either alone or as a component of a detergent composition. The soiled item may a textile, a dish or a hard surface. Stains released or removed may comprise blood, cocoa milk, carrot baby food and rice starch.

In one embodiment of the invention, the time period of a wash cycle is reduced by at least 25% when compared to bench mark wash. The time period may be reduced by at least 30%, by at least 35%, by at least 40%, by at least 45%, by at least 50%, by at least 55%, by at least 60%, by at least 62.5% or by at least 65% when compared to bench mark wash.

In one embodiment, the time period is reduced by at least 30 minutes, by at least 35 minutes, by at least 40 minutes, by at least 45 minutes, by at least 50 minutes, by at least 55 minutes, by at least 60 minutes, by at least 65 minutes, by at least 70 minutes, by at least 75 minutes, by at least 80 minutes, by at least 85 minutes of by at least 90 minutes when compared to bench mark wash.

The temperature of the wash liquor is in the range of 5° C. to 95° C., or in the range of 10° C. to 80° C., or in the range of 10° C. to 70° C., or in the range of 10° C. to 60° C., or in the range of 10° C. to 50° C., or in the range of 15° C. to 40° C., or in the range of 20° C. to 30° C.

The washing method comprises exposing a textile is exposed to a wash liquor during a first and optionally a second and third wash cycle. The pH of the wash liquor may be in the range of 7-10, preferably 7 to 9 such as 7.5. After completing a wash cycle the textile may go through a rinse cycle e.g. by being exposed to clean water or clean water comprising a conditioner.

The textile can be any textile as defined herein, or it can be made of cotton, cotton/polyester blends or nylon.

In one embodiment of the invention, the delta remission value is at least 2, at least 3 or at least 4.

The invention further concerns an item treated according to the inventive method.

### Detergent Compositions

In one embodiment, the invention is directed to detergent compositions comprising the multi enzyme composition of the present invention in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

The non-limiting list of composition components illustrated hereinafter are suitable for use in the compositions and methods herein may be desirably incorporated in certain embodiments of the invention, e.g. to assist or enhance cleaning performance, for treatment of the substrate to be cleaned, or to modify the aesthetics of the composition as is the case with perfumes, colorants, dyes or the like. The levels of any such components incorporated in any compositions are in addition to any materials previously recited for incorporation. The precise nature of these additional components, and levels of incorporation thereof, will depend on the physical form of the composition and the nature of the cleaning operation for which it is to be used. Although components mentioned below are categorized by general header according to a particular functionality, this is not to be construed as a limitation, as a component may comprise additional functionalities as will be appreciated by the skilled artisan.

Unless otherwise indicated the amounts in percentage is by weight of the composition (wt %). Suitable component materials include, but are not limited to, surfactants, builders, chelating agents, dye transfer inhibiting agents, dispersants, enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, hueing dyes, perfumes, perfume delivery systems, structure elasticizing agents, fabric softeners, carriers, hydrotropes, processing aids, solvents and/or pigments. In addition to the disclosure below, suitable examples of such other components and levels of use are found in U.S. Pat. No. 5,576,282, U.S. Pat. No. 6,306,812, and U.S. Pat. No. 6,326,348 hereby incorporated by reference.

Thus, in certain embodiments the invention do not contain one or more of the following adjuncts materials: surfactants, soaps, builders, chelating agents, dye transfer inhibiting agents, dispersants, additional enzymes, enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, perfume delivery systems, structure elasticizing agents, fabric softeners, carriers, hydrotropes, processing aids, solvents and/or pigments. However, when one or more components are present, such one or more components may be present as detailed below:

### Additional Enzymes

The multi enzyme composition as well as the detergent composition may comprise one or more additional enzymes such as a protease, lipase, cutinase, an amylase, carbohydrase, cellulase, pectinase, mannanase, arabinase, galactanase, pectate lyase, xylanase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases:

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include Celluzyme™, and Carezyme™ (Novozymes NS), Clazinase™, and Puradax HA™ (Genencor International Inc.), and KAC-500(B)™ (Kao Corporation).

Lyases:

The lyase may be a pectate lyase derived from Bacillus, particularly B. licherniformis or B. agaradhaerens, or a variant derived of any of these, e.g. as described in U.S. Pat. No. 6,124,127, WO 99/027083, WO 99/027084, WO 02/006442, WO 02/092741, WO 03/095638, A commercially available pectate lyase is XPect, Pectawash and Pectaway (Novozymes A/S).

Mannanase:

The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 99/064619. A commercially available mannanase is Mannaway (Novozymes A/S).

Proteases:

Suitable proteases include those of animal, vegetable or microbial origin. Microbial origin is preferred. Chemically modified or protein engineered mutants are included. The protease may be a serine protease or a metalloprotease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g., of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235, and 274.

Preferred commercially available protease enzymes include Alcalase™, Savinase™ Primase™, Duralase™, Esperase™, and Kannase™ (Novozymes A/S), Maxatase™, Maxacal™ Maxapem™, Properase™, Purafect™, Purafect OxP™, FN2™, and FN3™ (Genencor International Inc.).

Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615,

WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the 554V variant used in the commercial product Gentle Power Bleach from Huntsman Textile Effects Pte Ltd (WO10/100028).

Amylases:

Suitable amylases which can be used together with XX of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 3 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™ Purastar™, and Powerase (from Genencor International Inc.).

Peroxidases/Oxidases:

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ (Novozymes A/S).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein, the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein, the detergent will usually contain from about from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein, the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in laundry/ADW/hard surface cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PH DA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

The detergent may contain 0-30% by weight, such as about 1% to about 20%, of a bleaching system. Any bleaching system known in the art for use in laundry/ADW/hard surface cleaning detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

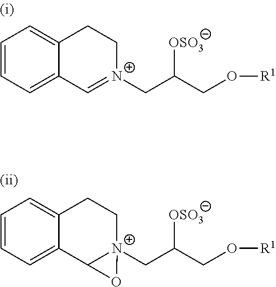

(iii) and mixtures thereof;

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Form of the Composition

The compositions described herein are advantageously employed for example, in laundry applications, hard surface cleaning, dishwashing applications, as well as cosmetic applications such as dentures, teeth, hair and skin. The compositions of the invention are in particular solid or liquid cleaning and/or treatment compositions. In one aspect the invention relates to a composition, wherein the form of the composition is selected from the group consisting of a regular, compact or concentrated liquid; a gel; a paste; a soap bar; a regular or a compacted powder; a granulated solid; a homogenous or a multilayer tablet with two or more layers (same or different phases); a pouch having one or more compartments; a single or a multi-compartment unit dose form; or any combination thereof.

The form of the composition may separate the components physically from each other in compartments such as e.g. water dissolvable pouches or in different layers of tablets. Thereby negative storage interaction between components can be avoided. Different dissolution profiles of each of the compartments can also give rise to delayed dissolution of selected components in the wash solution.

Pouches can be configured as single or multi compartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids (US2009/0011970 A1).

Water-Soluble Film—

The compositions of the present invention may also be encapsulated within a water-soluble film. Preferred film materials are preferably polymeric materials. The film material can e.g. be obtained by casting, blow-moulding, extrusion or blown extrusion of the polymeric material, as known in the art. Preferred polymers, copolymers or derivatives thereof suitable for use as pouch material are selected from polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, natural gums such as xanthum and carragum. More preferred polymers are selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and most preferably selected from polyvinyl alcohols, polyvinyl alcohol copolymers and hydroxypropyl methyl cellulose (HPMC), and combinations thereof. Preferably, the level of polymer in the pouch material, e.g. a PVA polymer, is at least 60 wt %. The polymer can have any weight average molecular weight, preferably from about 1.000 to 1.000.000, from about 10.000 to 300.000, from about 20.000 to 150.000. Mixtures of polymers can also be used as the pouch material.

Naturally, different film material and/or films of different thickness may be employed in making the compartments of the present invention. A benefit in selecting different films is that the resulting compartments may exhibit different solubility or release characteristics.

Preferred film materials are PVA films known under the MonoSol trade reference M8630, M8900, H8779 and those described in U.S. Pat. No. 6,166,117 and U.S. Pat. No. 6,787,512 and PVA films of corresponding solubility and deformability characteristics.

The film material herein can also comprise one or more additive ingredients. For example, it can be beneficial to add plasticisers, e.g. glycerol, ethylene glycol, diethyleneglycol, propylene glycol, sorbitol and mixtures thereof. Other additives include functional detergent additives to be delivered to the wash water, e.g. organic polymeric dispersants, etc.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids. Ref: (US2009/0011970 A1).

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The multi enzyme composition of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, the multi enzyme composition, optionally one or more additional enzymes, a protease inhibitor, and a salt of a monovalent cation and an organic anion may be prepared and the mixture is then plodded. The multi enzyme composition and optional additional enzymes may be added at the same time as the protease inhibitor for example in liquid form. Besides the mixing step and the plodding step, the process may further comprise the steps of milling, extruding, cutting, stamping, cooling and/or wrapping.

### Granular Detergent Formulations

A granular detergent may be formulated as described in WO09/092699, EP1705241, EP1382668, WO07/001262, U.S. Pat. No. 6,472,364, WO04/074419 or WO09/102854. Other useful detergent formulations are described in WO09/124162, WO09/124163, WO09/117340, WO09/117341, WO09/117342, WO09/072069, WO09/063355, WO09/132870, WO09/121757, WO09/112296, WO09/112298, WO09/103822, WO09/087033, WO09/050026, WO09/047125, WO09/047126, WO09/047127, WO09/047128, WO09/021784, WO09/010375, WO09/000605, WO09/122125, WO09/095645, WO09/040544, WO09/040545, WO09/024780, WO09/004295, WO09/004294, WO09/121725, WO09/115391, WO09/115392, WO09/074398, WO09/074403, WO09/068501, WO09/065770, WO09/021813, WO09/030632, WO09/015951, WO2011025615, WO2011016958, WO2011005803, WO2011005623, WO2011005730, WO2011005844, WO2011005904, WO2011005630, WO2011005830, WO2011005912, WO2011005905, WO2011005910, WO2011005813, WO2010135238, WO2010120863, WO2010108002, WO2010111365, WO2010108000, WO2010107635, WO2010090915, WO2010033976, WO2010033746, WO2010033747, WO2010033897, WO2010033979, WO2010030540, WO2010030541, WO2010030539, WO2010024467, WO2010024469, WO2010024470, WO2010025161, WO2010014395, WO2010044905, WO2010145887, WO2010142503, WO2010122051, WO2010102861, WO2010099997, WO2010084039, WO2010076292, WO2010069742, WO2010069718, WO2010069957, WO2010057784, WO2010054986, WO2010018043, WO2010003783, WO2010003792, WO2011023716, WO2010142539, WO2010118959, WO2010115813, WO2010105942, WO2010105961, WO2010105962, WO2010094356, WO2010084203, WO2010078979, WO2010072456, WO2010069905, WO2010076165, WO2010072603, WO2010066486, WO2010066631, WO2010066632, WO2010063689, WO2010060821, WO2010049187, WO2010031607, WO2010000636,

### Processes of Making the Compositions

The compositions of the present invention can be formulated into any suitable form and prepared by any process chosen by the formulator, non-limiting examples of which are described in Applicants' examples and in U.S. Pat. No. 4,990,280; US20030087791A1; US20030087790A1; US20050003983A1; US20040048764A1; U.S. Pat. No. 4,762,636; U.S. Pat. No. 6,291,412; US20050227891A1; EP1070115A2; U.S. Pat. No. 5,879,584; U.S. Pat. No. 5,691,297; U.S. Pat. No. 5,574,005; U.S. Pat. No. 5,569,645; U.S. Pat. No. 5,565,422; U.S. Pat. No. 5,516,448; U.S. Pat. No. 5,489,392; U.S. Pat. No. 5,486,303 all of which are incorporated herein by reference. The compositions of the invention or prepared according to the invention comprise cleaning and/or treatment composition including, but not limited to, compositions for treating fabrics, hard surfaces and any other surfaces in the area of fabric and home care, including: air care including air fresheners and scent delivery systems, car care, dishwashing, fabric conditioning (including softening and/or freshening), laundry detergency, laundry and rinse additive and/or care, hard surface cleaning and/or treatment including floor and toilet bowl cleaners, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use: car or carpet shampoos, bathroom cleaners including toilet bowl cleaners; as well as cleaning auxiliaries such as bleach additives and “stain-stick” or pre-treat types, substrate-laden compositions such as dryer added sheets. Preferred are compositions and methods for cleaning and/or treating textiles and/or hard surfaces, most preferably textiles. The compositions are preferably compositions used in a pre-treatment step or main wash step of a washing process, most preferably for use in textile washing step.

As used herein, the term “fabric and/or hard surface cleaning and/or treatment composition” is a subset of cleaning and treatment compositions that includes, unless otherwise indicated, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, car or carpet shampoos, bathroom cleaners including toilet bowl cleaners; fabric conditioning compositions including softening and/or freshening that may be in liquid, solid and/or dryer sheet form; as well as cleaning auxiliaries such as bleach additives and “stain-stick” or pre-treat types, substrate-laden compositions such as dryer added sheets. All of such compositions which are applicable may be in standard, concentrated or even highly concentrated form even to the extent that such compositions may in certain aspect be non-aqueous.

### Method of Use

The present invention includes a method for cleaning any surface including treating a textile or a hard surface or other surfaces in the field of fabric and/or home care. In one aspect of the invention, the method comprises the step of contacting the surface to be treated in a pre-treatment step or main wash step of a washing process, most preferably for use in a textile washing step or alternatively for use in dishwashing including both manual as well as automated/mechanical dishwashing. In one embodiment of the invention the multi enzyme composition and other components are added sequentially into the method for cleaning and/or treating the surface. Alternatively, the multi enzyme composition and other components are added simultaneously.

As used herein, washing includes but is not limited to, scrubbing, and mechanical agitation. Washing may be conducted with a foam composition as described in WO08/101958 and/or by applying alternating pressure (pressure/vacuum) as an addition or as an alternative to scrubbing and mechanical agitation. Drying of such surfaces or fabrics may be accomplished by any one of the common means employed either in domestic or industrial settings. The cleaning compositions of the present invention are ideally suited for use in laundry as well as dishwashing applications. Accordingly, the present invention includes a method for cleaning an object including but not limiting to fabric, tableware, cutlery and kitchenware. The method comprises the steps of contacting the object to be cleaned with a said cleaning composition comprising at least one embodiment of Applicants' cleaning composition, cleaning additive or mixture thereof. The fabric may comprise most any fabric capable of being laundered in normal consumer or institutional use conditions. The solution may have a pH from 8 to 10.5. The compositions may be employed at concentrations from 500 to 15.000 ppm in solution. The water temperatures typically range from 5° C. to 90° C. The water to fabric ratio is typically from 1:1 to 30:1.

### Use in Detergents

The polypeptides of the present invention may be added to and thus become a component of a detergent composition.

The detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the present invention provides a detergent additive comprising a polypeptide of the present invention as described herein.

### Unit Dose

In one aspect, the automatic dishwashing detergent composition of the invention is in unit dose form. Automatic dishwashing detergent products in unit dose form include tablets, capsules, sachets, pouches, etc. In one aspect, for use herein are tablets wrapped with a water-soluble film and water-soluble pouches. The weight of the composition of the invention is from about 10 to about 25 grams, from about 12 to about 24 grams or even from 14 to 22 grams. These weights are extremely convenient for automatic dishwashing detergent product dispenser fit. In the cases of unit dose products having a water-soluble material enveloping the automatic dishwashing detergent composition, the water-soluble material is not considered as part of the composition. In one aspect, the unit dose form is a water-soluble pouch (i.e., water-soluble film enveloping an automatic dishwashing detergent composition), in one aspect, a multicompartment pouch having a plurality of films forming a plurality of compartments. This configuration contributes to the flexibility and optimization of the composition. It allows for the separation and controlled release of different ingredients. In one aspect, one compartment contains an automatic dishwashing detergent composition in solid form and another compartment contains an automatic dishwashing detergent composition in liquid form.

In one aspect, multi-compartment pouch embodiments two different compartments could contain two different cleaning agents. In one aspect, the films of these two compartments have different dissolution profiles, allowing the release of the same or different agents at different times. For example, the agent from one compartment (first compartment) can be delivered early in the washing process to help with soil removal and a second agent from another compartment (second compartment) can be delivered at least two minutes, or even at least five minutes later than the agent from the first compartment.

In one aspect, a multi-compartment pouch comprising two side-by-side compartments superposed onto another compartment wherein at least two different compartments contain two different automatic dishwashing detergent compositions is disclosed.

According to another aspect of the invention, there is provided an automatic dishwashing detergent dosing element for use in an auto-dosing device the dosing element comprising an automatic dishwashing detergent composition according to any of the preceding claims. By “auto-dosing device” herein is meant a device that is placed into the dishwasher holding a plurality of doses to be delivered in different washes. The user does not need to charge the detergent for each wash, the auto-dosing device delivers them automatically. Each wash can use a single or more doses.

A multi-compartments pack is formed by a plurality of water-soluble enveloping materials which form a plurality of compartments, one of the compartments would contain the automatic dishwashing detergent composition of the invention, another compartment can contain a liquid composition, the liquid composition can be aqueous (i.e. comprises more than 10 percent of water by weight of the liquid composition) and the compartment can be made of warm water soluble material. In some embodiments the compartment comprising the automatic dishwashing detergent composition of the invention is made of cold water soluble material. It allows for the separation and controlled release of different ingredients. In other embodiments all the compartments are made of warm water soluble material.

Suitable packs comprise at least two side-by-side compartments superposed (i.e. placed above) onto another compartment, especially suitable are pouches. This disposition contributes to the compactness, robustness and strength of the pack, additionally, it minimises the amount of water-soluble material required. It only requires three pieces of material to form three compartments. The robustness of the pack allows also for the use of very thin films without compromising the physical integrity of the pack. The pack is also very easy to use because the compartments do not need to be folded to be used in machine dispensers of fix geometry. At least two of the compartments of the pack contain two different automatic dishwashing detergent compositions. By “different compositions” herein is meant automatic dishwashing detergent compositions that differ in at least one ingredient.

In one aspect, at least one of the compartments contains a solid automatic dishwashing detergent composition and another compartment an aqueous liquid automatic dishwashing detergent composition, the compositions are typically in a solid to liquid weight ratio of from about 20:1 to about 1:20, from about 18:1 to about 2:1 or from about 15:1 to about 5:1. This kind of pack is very versatile because it can accommodate compositions having a broad spectrum of values of solid:liquid ratio. Pouches having a high solid:liquid ratio because many of the detergent ingredients are particularly suitable for use in solid form, in one aspect in powder form. The ratio solid:liquid defined herein refers to the relationship between the weight of all the solid compositions and the weight of all the liquid compositions in the pack.

Suitable solid:liquid weight ratios are from about 2:1 to about 18:1, or from about 5:1 to about 15:1. These weight ratios are suitable in cases in which most of the ingredients of the detergent are in liquid form.

In one aspect, the two side-by-side compartments contain liquid automatic dishwashing detergent compositions, which can be the same or different and another compartment contains a solid automatic dishwashing detergent composition, for example in powder form, in one aspect, a densified powder. The solid composition contributes to the strength and robustness of the pack.

For dispenser fit reasons, especially in an automatic dishwasher, the unit dose form products herein have a square or rectangular base and a height of from about 1 to about 5 cm, or from about 1 to about 4 cm. In one aspect, the weight of the solid composition is from about 5 to about 20 grams, or from about 10 to about 15 grams and the weight of the liquid compositions is from about 0.5 to about 4 grams, or from about 0.8 to about 3 grams. In one aspect, at least two of the films which form different compartments have different solubilities, under the same conditions. This enables the release of the compositions which they partially or totally envelope at different times.

Controlled release of the ingredients of a multi-compartment pouch can be achieved by modifying the thickness of the film and/or the solubility of the film material. The solubility of the film material can be delayed by for example cross-linking the film as described in WO 2002/102955. Other water-soluble films designed for rinse release are described in U.S. Pat. No. 4,765,916 and U.S. Pat. No. 4,972,017. Waxy coating (see U.S. Pat. No. 5,453,216) of films can help with rinse release. pH controlled release means are described in U.S. Pat. No. 5,453,216, in particular amino-acetylated polysaccharide having selective degree of acetylation.

Other means of obtaining delayed release by multi-compartment pouches with different compartments, where the compartments are made of films having different solubility are taught in U.S. Pat. No. 6,727,215.

### Uses

The present invention is also directed to methods for using the enzyme of the present invention or compositions thereof in house hold laundry washing or industry laundry washing. The present invention is also directed to the use of enzyme of the present invention or compositions thereof in hard surface cleaning such as cleaning of floor, tables, walls and the like. The present invention is also directed to the use of enzyme of the present invention or compositions thereof cleaning of plates, cups, glasses, bowls and cutlery.

Use in Detergents.

The polypeptides of the present invention may be added to and thus become a component of a detergent composition.

The detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the present invention provides a detergent additive comprising a polypeptide of the present invention as described herein.

The invention is further summarized in the below paragraphs:
1. Multi enzyme composition comprising at least the following enzymes: protease, amylase, lipase, mannanase, pectate lyase and cellulase.
2. Composition according to any of the preceding paragraphs, wherein the protease is of animal, vegetable or microbial origin.
3. Composition according to any of the preceding paragraphs, wherein the protease is chemically modified or protein engineered.
4. Composition according to any of the preceding paragraphs, wherein the protease is a serine protease or a metalloprotease, preferably an alkaline microbial protease or a trypsin-like protease.
5. Composition according to any of the preceding paragraphs, wherein the protease is selected from the group consisting of Bacillus, e.g., subtilisin Novo, subtilisin Carlsberg, subtilisin 309, subtilisin 147, subtilisin 168, trypsin of bovine origin, trypsin of porcine origin and Fusarium protease.
6. Composition according to any of the preceding paragraphs, wherein the protease is an alkaline protease having at least 90% identity to the amino acid sequence of SEQ ID NO: 1 or a variant thereof with substitutions in one or more of the following positions: 27, 36, 57, 76, 87, 97, 101, 104, 120, 123, 167, 170, 194, 206, 218, 222, 224, 235, and 274, preferably the variant is an alkaline protease having at least 90% identity to the amino acid sequence of SEQ ID NO: 1 with the following substitution: M222S.
7. Composition according to any of the preceding paragraphs, wherein the amylase is an alpha-amylase or a glucoamylase.
8. Composition according to any of the preceding paragraphs, wherein the amylase is of bacterial or fungal origin.
9. Composition according to any of the preceding paragraphs, wherein the amylase is an alpha-amylase obtained from Bacillus, such as Bacillus licheniformis.
10. Composition according to any of the preceding paragraphs, wherein the amylase is an alpha-amylase having SEQ ID NO: 2 or a variant thereof having at least 80%, at least 85% or at least 90% sequence identity to SEQ ID NO: 2 and having a substitution, a deletion or an insertion of one amino acids downstream for the amino acid corresponding to the positions in the amylase having SEQ ID NO: 2: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484; Particular preferred amylases include such a variant having a deletion of D183 and G184 and having the substitutions R118K, N195F, R320K and R458K and a variant additionally having substitutions in one or more positions selected from the group: M9, G149, G182, G186, M202, T257, Y295, N299, M323, A339 and E345, most preferred a variant additionally having substitutions in all these positions; or a variant alpha-amylase derived from a parent α-amylase derived from B. licheniformis comprising the mutation: A1*+N2*+L3V+M15T+R23K+S29A+A30E+Y31H+A33S+E34D+H35I+M197T;
11. Composition according to any of the preceding paragraphs, wherein the wherein the lipase is of bacterial or fungal origin.
12. Composition according to any of the preceding paragraphs, wherein the lipase is chemically modified or protein engineered.
13. Composition according to any of the preceding paragraphs, wherein the lipase is derived from Thermomyces, e.g. from T. lanuginosus, cutinase from Humicola, e.g. H. insolens, strains of Pseudomonas, e.g. P. alcaligenes or P. pseudoalcaligenes, P. cepacia, P. sp. strain SD705, P. wisconsinensis, GDSL-type Streptomyces lipases, cutinase from Magnaporthe grisea, cutinase from Pseudomonas mendocina, Thermobifida fusca, Geobacillus stearothermophilus, Bacillus subtilis, Streptomyces griseus and S. pristinaespiralis.
14. Composition according to any of the preceding paragraphs, wherein the lipase is a polypeptide having an amino acid sequence which:
   a) has at least 90% identity with the wild-type lipase derived from Humicola lanuginosa strain DSM 4109;
   b) compared to said wild-type lipase, comprises a substitution of an electrically neutral or negatively charged amino acid at the surface of the three-dimensional structure within 15 Å of E1 or Q249 with a positively charged amino acid; and
   c) comprises a peptide addition at the C-terminal; and/or
   d) meets the following limitations:
   i. comprises a negative amino acid in position E210 of said wild-type lipase;
   ii. comprises a negatively charged amino acid in the region corresponding to positions 90-101 of said wild-type lipase; and
   iii. comprises a neutral or negative amino acid at a position corresponding to N94 of said wild-type lipase and/or has a negative or neutral net electric charge in the region corresponding to positions 90-101 of said wild-type lipase.
15. Composition according to any of the preceding paragraphs, wherein the mannanase has a sequence that is at least 80%, at least 85% or at least 90% identical to SEQ ID NO: 3
16. Composition according to any of the preceding paragraphs, wherein the pectate lyase is a variant of a parent enzyme having pectate lyase activity (EC 4.2.2.2) and comprising an alteration at one or more positions selected from the group consisting of positions number: 5, 9, 11, 26, 28, 30, 31, 37, 40, 45, 46, 47, 48, 49, 50, 51, 52, 54, 61, 64, 68, 69, 70, 71, 74, 75, 76, 79, 86, 87, 91, 99, 105, 106, 107, 111, 115, 116, 118, 122, 123, 134, 136, 139, 140, 141, 146, 148, 156, 158, 170, 182, 185, 186, 189, 193, 194, 196, 199, 201, 202, 204, 213, 215, 218, 224, 228, 229, 234, 235, 237, 251, 256, 257, 258, 272, 277, 286, 295, 298, 301, 302, 303, 305, 307, 308, 314, 316, 323, 324, 326, 331, 332, 333, 334, 335, 336, 337, 338, 339, 340, 341, 349, 356, 357, 363, 366, 378, 381, 384, 386, 387, 389, 390, 391, 393 and 397, wherein the alteration(s) are independently
   a. an insertion of an amino acid downstream of the amino acid which occupies the position,
   b. a deletion of the amino acid which occupies the position, or
   c. a substitution of the amino acid which occupies the position with a different amino acid,
and wherein each position corresponds to a position of the amino acid sequence of the pectate lyase having the amino acid sequence of SEQ ID NO:4, and wherein the parent enzyme is the pectate lyase shown on SEQ ID NO:4 or a pectate lyase having at least 70%, preferably at least 80%, more preferably at least 90%, even more preferably at least 95%, and most preferably at least 97% identity to the amino acid sequence of SEQ ID NO:4.
17. Composition according to any of the preceding paragraphs, wherein the cellulase is comprised by the enzyme classification EC 3.2.1.4.
18. Composition according to any of the preceding paragraphs, wherein the cellulase is of bacterial or fungal origin.
19. Composition according to any of the preceding paragraphs, wherein the cellulase is chemically modified or protein engineered.
20. Composition according to any of the preceding paragraphs, wherein the cellulase is derived from the genera Bacillus, Pseudomonas, Humicola, Fusarium, Thielavia or Acremonium.
21. Composition according to any of the preceding paragraphs, wherein the cellulase is derived from Humicola insolens, Myceliophthora thermophila, Sporotrichum pulverulentum, Fusarium oxysporum, Trichoderma reesei, Thielavia terrestris, Acremonium sp., Acremonium sp. CBS 478.94, Macrophomina phaseolina CBS 281.96, Crinipellis scabella CBS 280.96, Volutella colletotrichoides or Sordaria fimicola ATCC 52644 or Bacillus SP-KSMS237.
22. Composition according to any of the preceding paragraphs, wherein the cellulase is an enzyme exhibiting endo-beta-1,4-glucanase activity (EC 3.2.1.4), which is selected from one of
   d) a polypeptide encoded by the DNA sequence of positions 1 to 2322 of SEQ ID NO:5;
   e) a polypeptide produced by culturing a cell comprising the sequence of SEQ ID NO:5 under conditions wherein the DNA sequence is expressed; or
   f) an endo-beta-1,4-glucanase enzyme having a sequence of at least 97% identity to the amino acid sequence of position 1 to position 773 of SEQ ID NO:6.
23. Composition according to any of the preceding paragraphs, wherein the ratio between the protease and the other enzymes is 5:1, 3:1 or 2:1.
24. Detergent composition comprising a surfactant and multi enzyme composition according to any of paragraphs 1 to 23.
25. Detergent composition according to paragraph 24, wherein the composition further comprises: hydrotropes, builders, co-builders, a bleaching systems, polymers, fabric hueing agents, mediators and/or enzymes.
26. Detergent composition according to any of paragraphs 24-25, wherein the amount of surfactant is below 40% w/w of detergent composition, below 35% w/w of detergent composition, below 30% w/w of detergent composition, below 25% w/w of detergent composition or below 20% w/w of detergent composition.
27. Detergent composition according to any of paragraphs 24-26, wherein composition comprises at least one enzyme in addition to the multi enzyme composition.
28. Detergent composition according to any of paragraphs 24-27, wherein the additional enzyme is selected from the group consisting of protease, lipase, cutinase, amylase, carbohydrase, cellulase, pectate lyase, pectinase, mannanase, arabinase, galactanase, and/or xylanase.
29. Detergent composition according to any of paragraphs 24-28, wherein the concentration of each of the enzymes are in the range of 0.01-3.0% w/w enzyme of the detergent composition, in the range of 0.05-2.5% w/w enzyme of the detergent composition, in the range of 0.1-2.1% w/w enzyme of the detergent composition, in the range of 0.1-2.2% w/w enzyme of the detergent composition, in the range of 0.1-2.1% w/w enzyme of the detergent composition, in the range of 0.05-2.0% w/w enzyme of the detergent composition.
30. Detergent composition according to any of paragraphs 24-29, wherein the concentration of each of the enzymes are
   a. From 0.5-3.0% protease,
   b. From 0.01-1.5% amylase,
   c. From 0.01-1.0% lipase,
   d. From 0.01-1.0% mannanase,
   e. From 0.01-0.9% pectate lyase and
   f. From 0.01-1.0% cellulase
   calculated as % w/w enzyme of the detergent composition
31. Detergent composition according to any of paragraphs 24-30, wherein the concentration of each of the enzymes are
   a. From 1.5-2.5% protease,
   b. From 0.1-0.9% amylase,
   c. From 0.1-0.8% lipase,
   d. From 0.1-0.8% mannanase,
   e. From 0.1-0.5% pectate lyase and
   f. From 0.1-0.8% cellulase
   calculated as % w/w enzyme of the detergent composition
32. Detergent composition according to any of paragraphs 24-30, wherein the concentration of each of the enzymes are
   a. 2% protease,
   b. 0.5% amylase,
   c. 0.3% lipase,
   d. 0.3% mannanase,
   e. 0.2% pectate lyase and
   f. 0.3% cellulase
calculated as % w/w enzyme of the detergent composition.
33. Detergent composition according to any of paragraphs 24-32, wherein the composition is a bar, a homogenous tablet, a tablet having two or more layers, a pouch having one or more compartments, a regular or compact powder, a granule, a paste, a gel, or a regular, compact or concentrated liquid.
34. Detergent composition according to any of paragraphs 24-33, wherein the composition is a laundry cleaning composition, a dishwashing cleaning composition, a hard-surface cleaning composition and/or a personal care cleaning composition.
35. Use of a multi enzyme composition according to any of paragraphs 1-23 or a detergent composition according to any of paragraphs 24-34 for reducing the time period of a wash cycle for washing a soiled item.
36. Use according to paragraph 35, wherein the soiled item is a textile, a dish or a hard surface.
37. Use of a multi enzyme composition according to any of paragraphs 1-23 or a detergent composition according to any of paragraphs 24-34 for maintaining or improving whiteness of a textile.
38. Use of a multi enzyme composition according to any of paragraphs 1-23 or a detergent composition according to any of paragraphs 24-34 for reducing redeposition of soil on textiles.
39. Use of a multi enzyme composition according to any of paragraphs 1-23 or a detergent composition according to any of paragraphs 24-34 for removing or releasing the constituents of a skin-derived body soil deposition from a textile having a skin-derived body soil deposition.
40. Use according to paragraph 39, wherein yellowing is reduced.
41. Use according to any of paragraphs 35-36, wherein the time period is reduced by at least 25% when compared to a bench mark wash.
42. Use according to any of paragraphs 35-36 and 41, wherein the time period is reduced by at least 30%, by at least 35%, by at least 40%, by at least 45%, by at least 50%, by at least 55%, by at least 60%, by at least 62.5% or by at least 65%, or by at least 70%, or by at least 75% when compared to a bench mark wash.
43. Use according to any of paragraphs 35-36 and 41-42, wherein the time period is reduced by at least 30 minutes, by at least 35 minutes, by at least 40 minutes, by at least 45 minutes, by at least 50 minutes, by at least 55 minutes, by at least 60 minutes, by at least 65 minutes, by at least 70 minutes, by at least 75 minutes, by at least 80 minutes, by at least 85 minutes of by at least 90 minutes when compared to a bench mark wash.
44. Use according to any of the preceding use paragraphs, wherein the temperature is in the range of 5° C. to 95° C., or in the range of 10° C. to 80° C., or in the range of 10° C. to 70° C., or in the range of 10° C. to 60° C., or in the range of 10° C. to 50° C., or in the range of 15° C. to 40° C., or in the range of 20° C. to 30° C.
45. Use according to any of the preceding use paragraphs, wherein the delta remission value is at least 2, at least 3 or at least 4.
46. Use according to any of the preceding use paragraphs, wherein the amount of surfactant in a detergent composition is reduced compared to benchmark detergent compositions.
47. Use according to any of the preceding use paragraphs, wherein the amount of surfactant is reduced by at least 10%, or by at least 20%, or by at least 30% or by at least 40% compared to benchmark detergent compositions.
48. Method for reducing the time period of a wash cycle for washing a soiled item, wherein the item is exposed to a wash liquor comprising a multi enzyme composition according to any of paragraphs 1-23 or a detergent composition according to any of paragraphs 24-34.
49. Method according to paragraph 48, wherein the soiled item is a textile, a dish or a hard surface.
50. Method for maintaining or improving the whiteness of a textile wherein a textile is exposed to a wash liquor comprising a multi enzyme composition according to any of paragraphs 1-23 or a detergent composition according to any of paragraphs 24-34.
51. Method reducing redeposition of soil on a textile wherein a textile is exposed to a wash liquor comprising a multi enzyme composition according to any of paragraphs 1-23 or a detergent composition according to any of paragraphs 24-34.
52. Method for removing or releasing the constituents of a skin-derived body soil deposition from a textile having a skin-derived body soil deposition wherein a textile is exposed to a wash liquor comprising a multi enzyme composition according to any of paragraphs 1-23 or a detergent composition according to any of paragraphs 24-34.
53. Method according to paragraph 52, wherein yellowing is reduced.
54. Method according to any of paragraphs 48-49, wherein the time period is reduced by at least 25% when compared to a bench mark wash.
55. Method according to any of paragraphs 48-49 and 54, wherein the time period is reduced by at least 30%, by at least 35%, by at least 40%, by at least 45%, by at least 50%, by at least 55%, by at least 60%, by at least 62.5% or by at least 65% when compared to a bench mark wash.
56. Method according to any of paragraphs 48-49 and 54-55, wherein the time period is reduced by at least 30 minutes, by at least 35 minutes, by at least 40 minutes, by at least 45 minutes, by at least 50 minutes, by at least 55 minutes, by at least 60 minutes, by at least 65 minutes, by at least 70 minutes, by at least 75 minutes, by at least 80 minutes, by at least 85 minutes of by at least 90 minutes when compared to a bench mark wash.
57. Method according to any of the preceding method paragraphs, wherein the temperature is in the range of 5° C. to 95° C., or in the range of 10° C. to 80° C., or in the range of 10° C. to 70° C., or in the range of 10° C. to 60° C., or in the range of 10° C. to 50° C., or in the range of 15° C. to 40° C. or in the range of 20° C. to 30° C.
58. Method according to any of the preceding method paragraphs, wherein the delta remission value is at least 2, at least 3 or at least 4.
59. Method according to any of the preceding method paragraphs, wherein the textile is made of cotton, cotton/polyester or nylon.
60. Method according to any of the preceding method paragraphs, wherein the pH of the wash liquor is in the range of 7 to 10, preferably 7 to 9 such as 7.5.
61. Method according to any of the preceding method paragraphs, wherein stains comprising blood, cocoa milk, carrot baby food and rice starch are released or removed.
62. Method according to any of the preceding method paragraphs, wherein the textile is exposed to a wash liquor during a first and optionally a second and a third wash cycle.
63. Method according to any of the preceding method paragraphs, wherein the item is rinsed after being exposed to the wash liquor.
   Item treated according to the method of paragraphs 48-63.

### EXAMPLES

### Evaluation of Stains

Wash performance is expressed as a remission value (Rem). After washing and rinsing the swatches were spread out flat and allowed to air dry at room temperature overnight. All washes are evaluated the day after the wash. Light reflectance evaluations of the swatches were done using a Macbeth Color Eye 7000 reflectance spectrophotometer with very small aperture. The measurements were made without UV in the incident light and remission at 460 nm was extracted. Measurements were made on unwashed and washed swatches. The test swatch to be measured was placed on top of another swatch of same type and colour (twin swatch). With only one swatch of each kind per beaker, a swatch from a replicate wash was used in this way. Remission values for individual swatches were calculated by subtracting the remission value of the unwashed swatch from the remission value of the washed swatch. The total wash performance for each stained swatch set was calculated as the sum of individual Rem.

Calculating the enzyme effect is done by taking the remission measurements from the washed swatches. The total enzyme performance is calculated as the sum of individual remission measurements.

### Example 1

### Reducing the Washing Time and the Amount of Surfactant

The formulations were prepared according to table 1 below:

TABLE 1
Detergent formulations used

| No. | Detergent composition | Enzyme addition |
|---|---|---|
| Formulation | Model detergent B | No enzymes added |
| 1 | consisting of: 7% LAS | |
| | 3% AEOS/SLES - alkyl | |
| | ethoxylates/Sodium lauryl | |
| | ether sulfate | |
| | 6.6% Non-ionic surfactant | |
| | 5.5% soap | |
| | Add up 100% with water | |
| | Surfactant content: 16.6% | |
| Formulation | Model detergent B | Protease: 2 w/w % |
| 2 | consisting of: | Savinase 16 L |
| | 7% LAS | Amylase: 0.5 w/w % |
| | 3% AEOS/SLES alkyl | Stainzyme 12 L |
| | ethoxylates/Sodium lauryl | Lipase: 0.3 w/w % |
| | ether sulfate | Lipex 100 L |
| | 6.6% NI Non-ionic | Mannanase: 0.3 w/w % |
| | surfactant | Mannaway 4L |
| | 5.5% soap | Pectate lyase: 0.2 w/w % |
| | Add up to 100% water | Xpect 1000 L |
| | Surfactant content: 16.6% | Cellulase: 0.3 w/w % |
| | | Celluclean 4500 L |
| Formulation | Model detergent A | Protease: 1 w/w % |
| 3 | consisting of: | Savinase 16 L |
| | 12% LAS | Amylase: 0.1 w/w % |
| | 5% AEOS/SLES alkyl | Stainzyme 12 L |
| | ethoxylates/Sodium lauryl | |
| | ether sulfate | |
| | 11% Non-ionic surfactant | |
| | 5.5% soap | |
| | Add up to 100% water | |
| | Surfactant content: 28% | |

The wash liquors were used in washing processes (all at 15° dh) in the Wascator FOM7ICLS washing machine with varying wash times from 30 and up to 120 min and at various temperatures, 20° C. or 40° C. In each wash a broad mix of in total 35 technical stains (Double determination) were washed adding up to a total load of 3 kg with cotton and cotton/polyester ballast. After each wash there were two rinses irrespectively of the washing time. The formulations were dosed as of 50 g/wash corresponding to 3.85 g/L wash liquor. For all washes the following whiteness tracers were added: WFK 10A, WFK 20A, WFK 30A (WFK Testgewebe GmbH, Christenfeld 10, D-41379 Brüggen, Germany) and EMPA 221 (Swiss Federal Laboratoris for Materials Science and Technology, Überlandstrasse 129, 8600 Dübendorf, Switzerland).

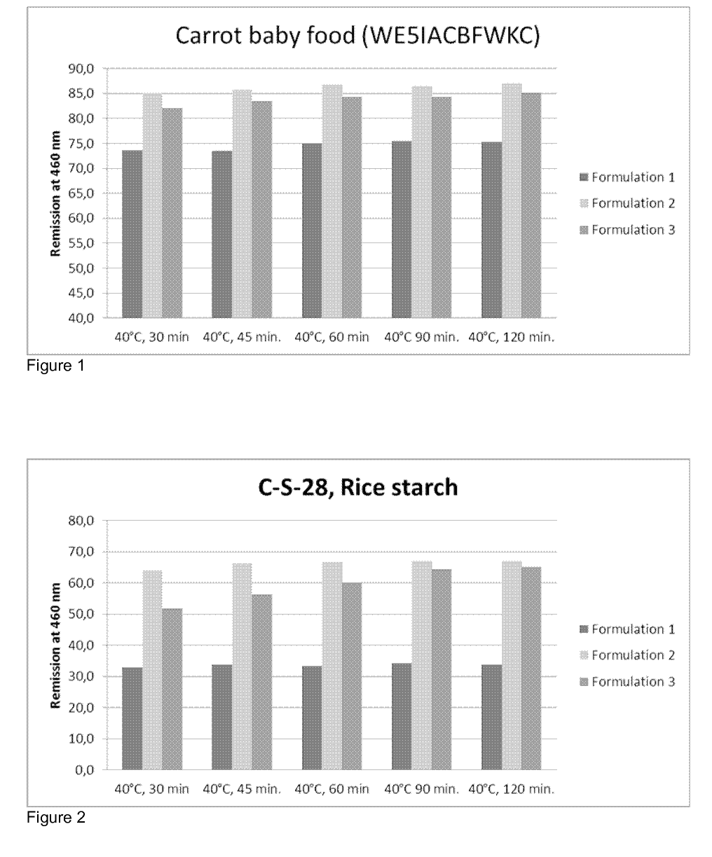

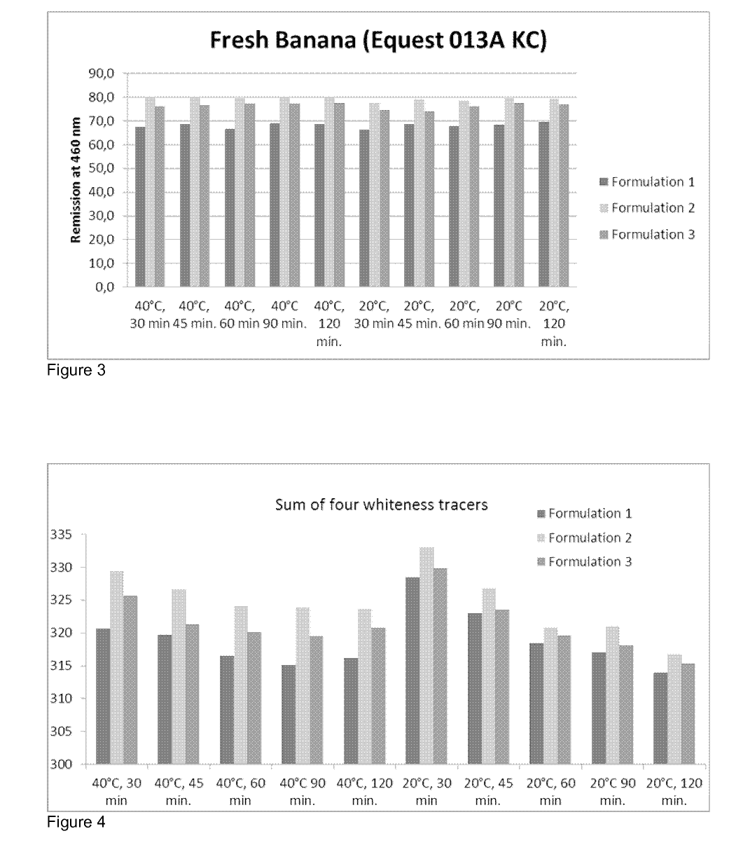

The results indicating the effect of reducing the washing time at a washing temperature of 40° C. at the same time maintaining the wash performance with a multi-enzyme containing formulation is shown in FIG. 1 and FIG. 2 for a single amylase-sensitive stains—Carrot baby food and Rice starch respectively—as well as in FIG. 3 for a Pectate lyase sensitive stain demonstrating the full washing effect already after 30 minutes of washing with multi-enzyme formulations and at both 20° C. and 40° C. The performance data is measured in terms of remission units being a way to quantify the solid removal (whiteness) on a given fabric. The higher the remission unit at 460 nm the greater is the soil removal and thus whiteness.

As observed from the FIGS. 1-3 when comparing the three formulations are the following:
There is a clear performance benefit of adding enzymes to the detergent bases tested as formulation 2 and formulation 3 have significantly higher wash performance (measured in terms of remission units at 460 nm) than formulation 1 (without enzymes)
Even with 40% reduced surfactant system the multi-enzyme formulation (formulation 2) outperforms the two other formulations in terms of wash performance as the bars for formulation 2 is significantly higher than for formulation 1 or formulation 3 at any given wash condition.
When comparing the washing time vs. performance data it is clear that the multi-enzyme composition (formulation 2) gives the maximum performance already after 30-45 minutes of washing time whereas the other formulations requires longer washing time to give their full—although notably lower—effect. This is observed as the bars for formulation 2 reaches a wash performance plateau much faster than for either of formulation 1 or 3.

TABLE 2
Raw data (remission unit numbers) corresponding to FIG. 1

| | 40° C., | 40° C., | 40° C., | 40° C., | 40° C., |
|---|---|---|---|---|---|
| | 30 min | 45 min | 60 min | 90 min | 120 min |
| Formulation 1 | 73.68 | 73.49 | 74.97 | 75.54 | 75.29 |
| Formulation 2 | 85.05 | 85.73 | 86.70 | 86.41 | 87.06 |
| Formulation 3 | 82.03 | 83.51 | 84.39 | 84.27 | 85.16 |

TABLE 3
Raw data (remission unit numbers) corresponding to FIG. 2

| | | 40° C., | 40° C., | 40° C., | 40° C., |
|---|---|---|---|---|---|
| | 40° C., 30 min | 45 min | 60 min | 90 min | 120 min |
| Formulation 1 | 32.96 | 33.79 | 33.47 | 34.20 | 33.81 |
| Formulation 2 | 63.89 | 66.18 | 66.61 | 66.80 | 66.98 |
| Formulation 3 | 51.88 | 56.28 | 60.14 | 64.34 | 65.20 |

TABLE 4
Raw data (remission unit numbers) corresponding to FIG. 3

| | | 40° C., | 40° C., | 40° C., | 40° C., |
|---|---|---|---|---|---|
| | 40° C., 30 min | 45 min | 60 min | 90 min | 120 min. |
| Formulation 1 | 67.62 | 68.75 | 66.52 | 68.87 | 68.78 |
| Formulation 2 | 80.05 | 80.29 | 79.66 | 80.08 | 80.29 |
| Formulation 3 | 76.16 | 76.74 | 77.26 | 77.28 | 77.61 |
| | | 20° C., | 20° C., | 20° C., | 20° C., |
| | 20° C., 30 min | 45 min | 60 min | 90 min | 120 min |
| Formulation 1 | 66.37 | 68.59 | 67.91 | 68.25 | 69.43 |
| Formulation 2 | 77.62 | 79.03 | 78.43 | 79.55 | 79.25 |
| Formulation 3 | 74.56 | 73.84 | 76.16 | 77.45 | 76.82 |

### Example 2

### Improving Whiteness—Less Reposition

The formulations were prepared as shown in table 1 in example 1.

The wash liquor were used in washing processes (all at 15° dh) in the Wascator

FOM7ICLS washing machine with varying wash times from 30 and up to 120 min and at various temperatures, 20° C. or 40° C. In each wash a broad mix of in total 35 AISE and non-AISE technical stains were washed (Double determination) adding up to a total load of 3 kg with cotton and cotton/polyester ballast. After each wash there were two rinses irrespectively of the washing time. The formulations were dosed as of 50 g/wash corresponding to 3.85 g/L wash liquor. For all washes the following whiteness tracers were added: WFK 10A, WFK 20A, WFK 30A and EMPA 221.

The results indicating the effect of reducing the washing time at a washing temperature of either 20° C. or 40° C. at the same time reducing the redeposition of removed soils from stains is shown in FIG. 4. The performance data is measured in terms of remission units being a way to quantify the solid removal (whiteness) on a given fabric. The higher the remission unit at 460 nm the greater is the soil removal and thus whiteness.

TABLE 5
Raw data (remission unit numbers) corresponding to FIG. 4

| | 40° C., | 40° C., | 40° C., | 40° C. | 40° C., | 20° C., | 20° C., | 20° C., | 20° C. | 20° C., |
|---|---|---|---|---|---|---|---|---|---|---|
| | 30 min | 45 min. | 60 min | 90 min. | 120 min. | 30 min | 45 min. | 60 min | 90 min. | 120 min. |
| Formulation 1 | 320.66 | 319.75 | 316.46 | 315.10 | 316.14 | 328.40 | 323.06 | 318.39 | 317.06 | 313.92 |
| Formulation 2 | 329.38 | 326.69 | 324.03 | 323.81 | 323.69 | 332.99 | 326.76 | 320.75 | 321.02 | 316.71 |
| Formulation 3 | 325.63 | 321.33 | 320.12 | 319.49 | 320.76 | 329.84 | 323.57 | 319.55 | 318.12 | 315.29 |

| | 20° C., | 20° C., | 20° C., | 20° C. | 20° C., |
|---|---|---|---|---|---|
| | 30 min. | 45 min. | 60 min. | 90 min. | 120 min. |
| Formulation 1 | 328.40 | 323.06 | 318.39 | 317.06 | 313.92 |
| Formulation 2 | 332.99 | 326.76 | 320.75 | 321.02 | 316.71 |
| Formulation 3 | 329.84 | 323.57 | 319.55 | 318.12 | 315.29 |

When comparing the performance of the three formulations in FIG. 4 the following trends become apparent:
The multi-enzyme solution (formulation 2) overall gives significantly greater remission units and thus greater whiteness for similar washing conditions when comparing the bars between formulation 2 and formulation 3 irrespectively of the washing condition
There are clear benefits in terms of whiteness of reducing the washing time irrespectively of washing temperature. This is apparent from FIG. 4 when comparing the height of the bars for all three formulations as a function of washing time. The longer washing time the longer the time for released soil to redeposit on the whiteness tracers.

Raw data (remission unit numbers) for all stains tested in example 1 and 2.

Red Wine

| | 20° C., | | 20° C., | | 20° C., | | 20° C., | | 20° C., | |
|---|---|---|---|---|---|---|---|---|---|---|
| | 30 min | | 45 min | | 60 min | | 90 min | | 120 min. | |
| | Rem. | std | Rem. | std | Rem. | std | Rem. | Std | Rem. | std |
| Formulation 1 | 64.11 | 1.04 | 64.71 | 1.13 | 64.62 | 0.38 | 64.37 | 0.71 | 64.53 | 1.55 |
| Formulation 2 | 64.23 | 0.39 | 64.32 | 0.86 | 64.62 | 0.96 | 65.20 | 1.24 | 65.70 | 0.98 |
| Formulation 3 | 64.36 | 0.86 | 64.57 | 0.77 | 64.47 | 1.24 | 64.08 | 1.07 | 63.46 | 1.45 |

| | | | | |
|---|---|---|---|---|
| 40° C., | 40° C., | 40° C., | 40° C. | 40° C., |
| 30 min | 45 min. | 60 min | 90 min. | 120 min. |

| Rem. | std | Rem. | std | Rem. | std | Rem. | std | Rem. | std |
|---|---|---|---|---|---|---|---|---|---|
| 66.20 | 0.54 | 69.18 | 0.19 | 63.11 | 1.21 | 63.94 | 0.75 | 61.80 | 0.72 |
| 66.37 | 0.56 | 69.34 | 0.55 | 63.88 | 0.93 | 64.22 | 0.38 | 63.33 | 1.65 |
| 67.75 | 0.86 | 70.70 | 0.53 | 64.30 | 1.54 | 65.71 | 0.43 | 64.48 | 1.30 |

Tomato puree

| | 20° C., | | 20° C., | | 20° C., | | 20° C., | | 20° C., | |
|---|---|---|---|---|---|---|---|---|---|---|
| | 30 min | | 45 min | | 60 min | | 90 min | | 120 min | |
| | Rem. | std | Rem. | std | Rem. | std | Rem. | Std | Rem. | Std |
| Formulation 1 | 53.40 | 2.57 | 55.33 | 2.01 | 53.06 | 0.42 | 59.19 | 1.53 | 61.19 | 3.30 |
| Formulation 2 | 60.02 | 1.62 | 68.17 | 2.53 | 67.37 | 2.29 | 74.76 | 3.59 | 76.84 | 1.42 |
| Formulation 3 | 59.51 | 1.03 | 61.01 | 2.55 | 63.65 | 0.74 | 65.88 | 2.62 | 71.44 | 3.61 |

| | | | | |
|---|---|---|---|---|
| 40° C., | 40° C., | 40° C., | 40° C. | 40° C., |
| 30 min | 45 min. | 60 min | 90 min. | 120 min. |

| Rem. | std | Rem. | std | Rem. | Std | Rem. | std | Rem. | std |
|---|---|---|---|---|---|---|---|---|---|
| 56.45 | 0.80 | 58.73 | 1.48 | 58.96 | 0.79 | 59.88 | 1.26 | 60.28 | 1.44 |
| 72.10 | 0.64 | 76.61 | 0.84 | 76.69 | 2.62 | 79.86 | 1.24 | 81.43 | 0.92 |
| 66.14 | 2.01 | 69.34 | 1.02 | 70.19 | 1.24 | 72.94 | 0.80 | 72.84 | 1.59 |

Carrot Baby food

| | 20° C., | | 20° C., | | 20° C., | | 20° C., | | 20° C., | |
|---|---|---|---|---|---|---|---|---|---|---|
| | 30 min | | 45 min | | 60 min | | 90 min | | 120 min | |
| | Rem. | std | Rem. | std | Rem. | std | Rem. | Std | Rem. | Std |
| Formulation 1 | 67.59 | 3.23 | 65.43 | 2.57 | 67.87 | 1.93 | 69.46 | 1.66 | 72.87 | 1.58 |
| Formulation 2 | 76.65 | 1.42 | 76.28 | 2.72 | 78.20 | 2.28 | 81.50 | 0.72 | 83.75 | 1.32 |
| Formulation 3 | 75.02 | 1.17 | 74.80 | 2.17 | 75.89 | 2.03 | 78.91 | 0.74 | 82.01 | 1.59 |

| | | | | |
|---|---|---|---|---|
| 40° C., | 40° C., | 40° C., | 40° C., | 40° C., |
| 30 min | 45 min | 60 min | 90 min | 120 min |

| Rem. | std | Rem. | std | Rem. | Std | Rem. | std | Rem. | Std |
|---|---|---|---|---|---|---|---|---|---|
| 73.68 | 1.31 | 73.49 | 0.87 | 74.97 | 2.58 | 75.54 | 2.65 | 75.29 | 1.16 |
| 85.05 | 1.40 | 85.73 | 0.76 | 86.70 | 0.54 | 86.41 | 0.15 | 87.06 | 0.52 |
| 82.03 | 0.97 | 83.51 | 0.58 | 84.39 | 0.59 | 84.27 | 0.26 | 85.16 | 1.16 |

French squeezy mustard

| | 20° C., | | 20° C., | | 20° C., | | 20° C., | | 20° C., | |
|---|---|---|---|---|---|---|---|---|---|---|
| | 30 min | | 45 min | | 60 min | | 90 min | | 120 min | |
| | Rem. | std | Rem. | Std | Rem. | std | Rem. | std | Rem. | Std |
| Formulation 1 | 27.77 | 0.29 | 28.98 | 1.02 | 29.77 | 0.66 | 33.80 | 0.61 | 36.05 | 1.33 |
| Formulation 2 | 28.52 | 0.83 | 31.22 | 1.32 | 32.17 | 2.04 | 33.52 | 0.33 | 37.91 | 1.11 |
| Formulation 3 | 29.13 | 0.53 | 31.72 | 1.14 | 35.56 | 1.39 | 36.81 | 0.45 | 39.65 | 1.60 |

| | | | | |
|---|---|---|---|---|
| 40° C., | 40° C., | 40° C., | 40° C., | 40° C., |
| 30 min | 45 min | 60 min | 90 min | 120 min |

| Rem. | std | Rem. | std | Rem. | std | Rem. | std | Rem. | Std |
|---|---|---|---|---|---|---|---|---|---|
| 35.29 | 0.77 | 35.92 | 0.87 | 38.60 | 0.58 | 41.13 | 0.86 | 42.60 | 1.29 |
| 35.10 | 0.89 | 38.46 | 1.12 | 40.85 | 0.85 | 43.43 | 1.38 | 47.23 | 0.73 |
| 37.80 | 1.32 | 40.63 | 1.26 | 42.61 | 1.48 | 45.32 | 2.03 | 48.84 | 1.07 |

| | Grass/Mud | | | | | | | | | |
|---|---|---|---|---|---|---|---|---|---|---|
| | 20° C., | | 20° C., 45 | | 20° C., | | 20° C., | | 20° C., | |
| | 30 min | | min | | 60 min | | 90 min | | 120 min | |
| | Rem. | std | Rem. | std | Rem. | std | Rem. | Std | Rem. | Std |
| Formulation 1 | 32.43 | 1.80 | 32.23 | 1.12 | 33.80 | 0.85 | 35.31 | 1.34 | 36.54 | 1.40 |
| Formulation 2 | 32.48 | 1.16 | 32.56 | 0.72 | 35.27 | 1.30 | 36.60 | 2.50 | 40.67 | 2.35 |
| Formulation 3 | 32.30 | 0.39 | 33.04 | 1.12 | 35.51 | 0.88 | 36.21 | 1.23 | 38.98 | 1.27 |

| | | 40° C., | | 40° C., | | 40° C. | | 40° C., | |
|---|---|---|---|---|---|---|---|---|---|
| 40° C., 30 min | | 45 min. | | 60 min | | 90 min. | | 120 min. | |
| Rem. | std | Rem. | std | Rem. | std | Rem. | std | Rem. | Std |
| 34.85 | 1.25 | 35.57 | 1.61 | 36.20 | 0.80 | 38.74 | 0.51 | 37.56 | 1.20 |
| 35.59 | 1.20 | 37.61 | 1.40 | 38.78 | 0.48 | 39.70 | 2.17 | 41.36 | 2.04 |
| 34.36 | 0.95 | 37.16 | 0.53 | 36.25 | 0.72 | 40.51 | 0.67 | 40.21 | 0.79 |

| | Blood | | | | | | | | | |
|---|---|---|---|---|---|---|---|---|---|---|
| | 20° C., | | 20° C., 45 | | 20° C., | | 20° C., | | 20° C., | |
| | 30 min | | min | | 60 min | | 90 min | | 120 min | |
| | Rem. | std | Rem. | std | Rem. | std | Rem. | Std | Rem. | Std |
| Formulation 1 | 44.72 | 1.74 | 46.29 | 1.30 | 46.07 | 0.45 | 46.23 | 0.12 | 45.92 | 0.79 |
| Formulation 2 | 50.40 | 1.01 | 54.85 | 0.50 | 54.66 | 0.75 | 55.31 | 0.43 | 54.87 | 0.38 |
| Formulation 3 | 51.58 | 1.67 | 52.82 | 0.55 | 53.58 | 0.50 | 53.51 | 0.66 | 54.99 | 0.71 |

| | | 40° C., | | 40° C., | | 40° C., | | 40° C., | |
|---|---|---|---|---|---|---|---|---|---|
| 40° C., 30 min | | 45 min | | 60 min | | 90 min | | 120 min | |
| Rem. | std | Rem. | std | Rem. | std | Rem. | std | Rem. | Std |
| 47.00 | 0.85 | 46.91 | 0.48 | 46.52 | 0.73 | 46.37 | 0.22 | 46.60 | 0.96 |
| 56.10 | 0.94 | 56.11 | 0.71 | 56.10 | 1.39 | 56.35 | 0.77 | 57.23 | 0.45 |
| 55.38 | 1.23 | 55.85 | 0.88 | 55.07 | 0.60 | 55.64 | 0.88 | 57.00 | 0.87 |

| | Fresh Banana | | | | | | | | | |
|---|---|---|---|---|---|---|---|---|---|---|
| | 20° C., | | 20° C., 45 | | 20° C., | | 20° C., | | 20° C., | |
| | 30 min | | min | | 60 min | | 90 min | | 120 min | |
| | Rem. | std | Rem. | std | Rem. | std | Rem. | Std | Rem. | Std |
| Formulation 1 | 66.37 | 2.93 | 68.59 | 1.43 | 67.91 | 1.38 | 68.25 | 1.82 | 69.43 | 0.77 |
| Formulation 2 | 77.62 | 0.95 | 79.03 | 1.04 | 78.43 | 0.64 | 79.55 | 0.62 | 79.25 | 1.39 |
| Formulation 3 | 74.56 | 1.87 | 73.84 | 0.66 | 76.16 | 0.84 | 77.45 | 0.68 | 76.82 | 0.85 |

| | | 40° C., | | 40° C., | | 40° C., | | 40° C., | |
|---|---|---|---|---|---|---|---|---|---|
| 40° C., 30 min | | 45 min | | 60 min | | 90 min | | 120 min | |
| Rem. | std | Rem. | std | Rem. | std | Rem. | std | Rem. | Std |
| 67.62 | 1.13 | 68.75 | 1.52 | 66.52 | 2.47 | 68.87 | 1.16 | 68.78 | 1.05 |
| 80.05 | 0.33 | 80.29 | 0.14 | 79.66 | 0.68 | 80.08 | 0.51 | 80.29 | 0.71 |
| 76.16 | 1.46 | 76.74 | 0.96 | 77.26 | 3.02 | 77.28 | 0.45 | 77.61 | 1.14 |

| | Tangarine | | | | | | | | | |
|---|---|---|---|---|---|---|---|---|---|---|
| | | | 20° C., | | | | | | 20° C., | |
| | 20° C., 30 | | 45 | | 20° C., | | 20° C., | | 120 | |
| | min | | min | | 60 min | | 90 min. | | min | |
| | Rem. | std | Rem. | std | Rem. | std | Rem. | std | Rem. | std |
| Formulation 1 | 48.85 | 4.50 | 52.29 | 6.40 | 49.87 | 1.80 | 56.31 | 6.90 | 60.40 | 7.41 |
| Formulation 2 | 60.35 | 2.45 | 63.74 | 4.01 | 64.59 | 2.47 | 67.81 | 2.31 | 72.06 | 5.77 |
| Formulation 3 | 57.69 | 4.07 | 57.19 | 2.37 | 60.06 | 2.19 | 61.05 | 4.57 | 67.92 | 5.37 |

| | | 40° C., | | 40° C., | | 40° C., | | | |
|---|---|---|---|---|---|---|---|---|---|
| 40° C., 30 min | | 45 min | | 60 min | | 90 min | | 40° C., 120 min | |
| Rem. | std | Rem. | std | Rem. | std | Rem. | std | Rem. | Std |
| 53.04 | 2.43 | 53.95 | 2.63 | 57.95 | 2.57 | 56.20 | 0.27 | 59.54 | 4.07 |
| 68.84 | 0.65 | 71.77 | 2.63 | 74.34 | 2.49 | 75.83 | 1.59 | 77.73 | 2.25 |
| 63.90 | 1.98 | 65.29 | 2.52 | 67.28 | 1.70 | 66.80 | 1.16 | 67.01 | 2.46 |

| | EMPA 101, Olive oil/carbon | | | | | | | | | |
|---|---|---|---|---|---|---|---|---|---|---|
| | 20° C., | | 20° C., 45 | | 20° C. | | 20° C., | | 20° C., | |
| | 30 min | | min | | 60 min | | 90 min | | 120 min | |
| | Rem. | std | Rem. | std | Rem. | std | Rem. | Std | Rem. | Std |
| Formulation 1 | 22.87 | 0.70 | 23.41 | 0.43 | 23.78 | 0.13 | 25.96 | 0.62 | 24.69 | 0.74 |
| Formulation 2 | 23.80 | 0.94 | 25.94 | 0.59 | 27.14 | 0.41 | 27.78 | 1.35 | 29.79 | 1.07 |
| Formulation 3 | 22.83 | 1.13 | 23.67 | 2.05 | 24.93 | 0.87 | 24.86 | 0.99 | 27.49 | 2.01 |

| | | 40° C., | | 40° C., | | 40° C. | | 40° C., | |
|---|---|---|---|---|---|---|---|---|---|
| 40° C., 30 min | | 45 min. | | 60 min | | 90 min. | | 120 min. | |
| Rem. | std | Rem. | std | Rem. | std | Rem. | std | Rem. | std |
| 23.96 | 0.46 | 24.64 | 0.62 | 24.59 | 0.39 | 25.23 | 0.92 | 25.77 | 1.59 |
| 27.29 | 0.20 | 27.42 | 0.53 | 28.41 | 0.65 | 29.30 | 0.93 | 31.80 | 1.37 |
| 24.28 | 0.69 | 25.82 | 0.68 | 25.93 | 1.22 | 28.07 | 0.73 | 28.17 | 0.47 |

| | EMPA 112, Cocoa milk | | | | | | | | | |
|---|---|---|---|---|---|---|---|---|---|---|
| | 20° C., | | 20° C., 45 | | 20° C., | | 20° C. | | 20° C., 120 | |
| | 30 min | | min. | | 60 min | | 90 min. | | min. | |
| | Rem. | std | Rem. | std | Rem. | std | Rem. | std | Rem. | Std |
| Formulation 1 | 36.28 | 0.71 | 36.97 | 0.81 | 37.74 | 1.35 | 38.41 | 1.46 | 38.72 | 0.44 |
| Formulation 2 | 47.18 | 3.16 | 47.70 | 1.01 | 51.05 | 1.78 | 51.53 | 1.10 | 51.82 | 1.24 |
| Formulation 3 | 43.11 | 1.81 | 44.53 | 1.47 | 48.31 | 2.34 | 47.94 | 0.70 | 50.53 | 0.88 |

| | | 40° C., | | 40° C., | | 40° C. | | 40° C., | |
|---|---|---|---|---|---|---|---|---|---|
| 40° C., 30 min | | 45 min. | | 60 min | | 90 min. | | 120 min. | |
| Rem. | std | Rem. | std | Rem. | std | Rem. | std | Rem. | std |
| 37.45 | 1.19 | 36.94 | 1.07 | 37.31 | 1.81 | 38.02 | 0.82 | 40.01 | 1.61 |
| 50.00 | 0.84 | 50.12 | 0.65 | 50.09 | 1.58 | 49.91 | 1.75 | 52.30 | 2.87 |
| 47.01 | 1.63 | 47.70 | 1.95 | 48.05 | 1.74 | 50.92 | 1.54 | 49.41 | 1.88 |

| | WFK 10D, Sebum/pigment | | | | | | | | | |
|---|---|---|---|---|---|---|---|---|---|---|
| | 20° C. | | 20° C., 45 | | 20° C., | | 20° C. | | 20° C., 120 | |
| | 30 min | | min. | | 60 min | | 90 min. | | min. | |
| | Rem. | std | Rem. | std | Rem. | std | Rem. | std | Rem. | std |
| Formulation 1 | 53.33 | 0.54 | 53.85 | 0.58 | 54.56 | 0.84 | 57.61 | 1.30 | 56.75 | 1.02 |
| Formulation 2 | 54.29 | 1.54 | 56.33 | 1.37 | 57.67 | 1.93 | 58.99 | 2.39 | 58.67 | 2.49 |
| Formulation 3 | 54.08 | 1.29 | 54.42 | 0.81 | 57.34 | 2.64 | 57.51 | 0.76 | 59.64 | 0.83 |

| 40° C., 30 min | | 40° C., 45 min. | | 40° C., 60 min | | 40° C. 90 min. | | 40° C., 120 min. | |
|---|---|---|---|---|---|---|---|---|---|
| Rem. | std | Rem. | std | Rem. | std | Rem. | std | Rem. | std |
| 55.75 | 1.09 | 56.91 | 1.29 | 57.15 | 1.23 | 57.48 | 1.78 | 59.47 | 1.35 |
| 58.06 | 0.83 | 57.36 | 1.94 | 59.20 | 2.36 | 59.71 | 1.58 | 62.47 | 1.41 |
| 56.59 | 0.66 | 56.77 | 2.09 | 57.35 | 1.48 | 61.15 | 0.65 | 60.04 | 1.04 |

| | WFK 20D, Sebum/pigment | | | | | | | | | |
|---|---|---|---|---|---|---|---|---|---|---|
| | | | 20° C., 45 | | | | | | 20° C., 120 | |
| | 20° C. 30 min | | min. | | 20° C., 60 min | | 20° C. 90 min. | | min. | |
| | Rem. | std | Rem. | std | Rem. | std | Rem. | std | Rem. | std |
| Formulation 1 | 57.08 | 0.97 | 58.87 | 1.12 | 60.08 | 1.62 | 63.12 | 0.84 | 63.81 | 2.36 |
| Formulation 2 | 59.20 | 0.80 | 63.99 | 0.44 | 65.01 | 0.93 | 67.41 | 0.92 | 66.77 | 1.17 |
| Formulation 3 | 59.91 | 1.19 | 62.36 | 1.01 | 64.89 | 1.83 | 66.54 | 0.90 | 67.71 | 1.14 |

| 40° C., 30 min | | 40° C., 45 min. | | 40° C., 60 min | | 40° C. 90 min. | | 40° C., 120 min. | |
|---|---|---|---|---|---|---|---|---|---|
| Rem. | std | Rem. | std | Rem. | std | Rem. | std | Rem. | std |
| 64.96 | 0.88 | 67.10 | 1.38 | 65.74 | 0.85 | 67.87 | 0.81 | 67.75 | 1.20 |
| 67.37 | 0.62 | 67.56 | 1.66 | 68.83 | 1.11 | 69.64 | 0.64 | 70.87 | 1.50 |
| 67.23 | 0.91 | 68.09 | 0.65 | 69.52 | 0.89 | 70.22 | 0.66 | 69.73 | 0.46 |

| | WFK 20MU, Make-up | | | | | | | | | |
|---|---|---|---|---|---|---|---|---|---|---|
| | | | 20° C., 45 | | | | | | 20° C., 120 | |
| | 20° C., 30 min | | min. | | 20° C., 60 min | | 20° C. 90 min. | | min. | |
| | Rem. | std | Rem. | std | Rem. | std | Rem. | std | Rem. | std |
| Formulation 1 | 63.99 | 0.61 | 64.56 | 1.10 | 65.12 | 1.12 | 65.93 | 1.08 | 66.95 | 1.96 |
| Formulation 2 | 64.10 | 1.83 | 66.07 | 1.09 | 66.20 | 0.97 | 67.52 | 0.61 | 67.36 | 1.03 |
| Formulation 3 | 64.50 | 1.57 | 65.56 | 1.16 | 66.32 | 2.10 | 67.62 | 1.17 | 68.44 | 0.89 |

| 40° C., 30 min | | 40° C., 45 min. | | 40° C., 60 min | | 40° C. 90 min. | | 40° C., 120 min. | |
|---|---|---|---|---|---|---|---|---|---|
| Rem. | std | Rem. | std | Rem. | std | Rem. | std | Rem. | std |
| 65.50 | 0.89 | 65.98 | 0.90 | 66.12 | 0.91 | 66.74 | 1.42 | 67.81 | 0.50 |
| 66.80 | 0.61 | 67.16 | 0.46 | 67.49 | 1.52 | 67.84 | 0.72 | 68.85 | 0.60 |
| 65.24 | 0.52 | 66.76 | 1.10 | 67.07 | 0.62 | 67.99 | 0.80 | 69.29 | 1.00 |

| | EMPA 164, Grass | | | | | | | | | |
|---|---|---|---|---|---|---|---|---|---|---|
| | | | 20° C., 45 | | | | | | 20° C., 120 | |
| | 20° C., 30 min | | min. | | 20° C., 60 min | | 20° C. 90 min. | | min. | |
| | Rem. | std | Rem. | std | Rem. | std | Rem. | std | Rem. | std |
| Formulation 1 | 36.75 | 0.92 | 36.84 | 0.91 | 37.16 | 0.84 | 39.16 | 0.63 | 41.64 | 1.22 |
| Formulation 2 | 40.40 | 1.92 | 43.14 | 0.90 | 44.82 | 0.62 | 45.34 | 1.19 | 47.90 | 2.11 |
| Formulation 3 | 43.13 | 0.35 | 44.41 | 1.04 | 47.80 | 2.72 | 46.41 | 0.44 | 50.71 | 0.89 |

| 40° C., 30 min | | 40° C., 45 min. | | 40° C., 60 min | | 40° C. 90 min. | | 40° C., 120 min. | |
|---|---|---|---|---|---|---|---|---|---|
| Rem. | std | Rem. | std | Rem. | std | Rem. | std | Rem. | std |
| 41.12 | 0.72 | 41.61 | 0.52 | 42.98 | 1.41 | 42.07 | 0.74 | 43.02 | 1.18 |
| 48.80 | 2.29 | 49.67 | 0.78 | 49.62 | 1.98 | 50.30 | 1.16 | 51.62 | 1.13 |
| 50.14 | 1.60 | 51.69 | 1.39 | 51.87 | 0.21 | 52.28 | 0.36 | 51.61 | 0.52 |

| | EMPA 117, Blood/Milk/Carbon | | | | | | | | | |
|---|---|---|---|---|---|---|---|---|---|---|
| | | | 20° C., 45 | | | | | | 20° C., 120 | |
| | 20° C., 30 min | | min. | | 20° C., 60 min | | 20° C. 90 min. | | min. | |
| | Rem. | std | Rem. | std | Rem. | std | Rem. | std | Rem. | std |
| Formulation 1 | 26.62 | 0.74 | 27.03 | 0.68 | 28.11 | 1.11 | 30.62 | 1.06 | 30.13 | 1.57 |
| Formulation 2 | 35.86 | 1.36 | 38.07 | 0.82 | 40.25 | 0.92 | 41.62 | 1.78 | 41.56 | 0.86 |
| Formulation 3 | 40.37 | 1.27 | 44.57 | 1.54 | 47.38 | 1.49 | 48.65 | 0.80 | 49.98 | 1.05 |

| 40° C., 30 min | | 40° C., 45 min. | | 40° C., 60 min | | 40° C. 90 min. | | 40° C., 120 min. | |
|---|---|---|---|---|---|---|---|---|---|
| Rem. | std | Rem. | std | Rem. | std | Rem. | std | Rem. | std |
| 28.09 | 0.98 | 29.47 | 0.92 | 29.82 | 0.79 | 28.92 | 1.12 | 31.10 | 1.59 |
| 36.16 | 4.44 | 37.02 | 1.02 | 37.79 | 3.30 | 38.34 | 0.96 | 40.95 | 1.61 |
| 44.48 | 0.69 | 46.82 | 1.06 | 47.24 | 0.69 | 48.74 | 1.74 | 48.68 | 2.86 |

| | EMPA 111, Blood | | | | | | | | | |
|---|---|---|---|---|---|---|---|---|---|---|
| | | | 20° C., 45 | | | | | | 20° C., 120 | |
| | 20° C., 30 min | | min. | | 20° C., 60 min | | 20° C. 90 min. | | min. | |
| | Rem. | std | Rem. | std | Rem. | std | Rem. | std | Rem. | std |
| Formulation 1 | 37.75 | 3.30 | 39.06 | 1.32 | 44.21 | 0.54 | 46.06 | 1.35 | 48.21 | 1.39 |
| Formulation 2 | 43.49 | 3.58 | 45.85 | 0.63 | 53.32 | 1.59 | 56.07 | 2.53 | 56.37 | 0.61 |
| Formulation 3 | 47.70 | 0.77 | 52.87 | 0.84 | 58.78 | 1.47 | 60.35 | 1.94 | 62.42 | 0.79 |

| 40° C., 30 min | | 40° C., 45 min. | | 40° C., 60 min | | 40° C. 90 min. | | 40° C., 120 min. | |
|---|---|---|---|---|---|---|---|---|---|
| Rem. | std | Rem. | std | Rem. | std | Rem. | std | Rem. | std |
| 39.21 | 1.76 | 41.34 | 0.88 | 39.66 | 1.29 | 43.14 | 0.84 | 41.75 | 1.20 |
| 49.41 | 1.47 | 51.13 | 1.16 | 51.55 | 0.70 | 58.09 | 1.63 | 57.93 | 1.35 |
| 53.89 | 2.38 | 56.19 | 2.57 | 59.10 | 3.02 | 63.87 | 0.49 | 64.05 | 2.39 |

| | C-S-28, Rice starch | | | | | | | | | |
|---|---|---|---|---|---|---|---|---|---|---|
| | | | 20° C., 45 | | | | | | 20° C., 120 | |
| | 20° C., 30 min | | min. | | 20° C., 60 min | | 20° C. 90 min. | | min. | |
| | Rem. | std | Rem. | std | Rem. | std | Rem. | std | Rem. | std |
| Formulation 1 | 30.13 | 0.74 | 30.91 | 0.77 | 31.39 | 0.62 | 32.27 | 0.76 | 32.18 | 1.27 |
| Formulation 2 | 37.99 | 1.43 | 40.85 | 1.17 | 44.80 | 1.13 | 47.66 | 0.96 | 50.26 | 1.24 |
| Formulation 3 | 33.36 | 0.95 | 34.63 | 1.56 | 36.59 | 0.83 | 37.86 | 0.98 | 41.58 | 1.40 |

| 40° C., 30 min | | 40° C., 45 min. | | 40° C., 60 min | | 40° C. 90 min. | | 40° C., 120 min. | |
|---|---|---|---|---|---|---|---|---|---|
| Rem. | std | Rem. | std | Rem. | std | Rem. | std | Rem. | std |
| 32.96 | 1.04 | 33.79 | 1.01 | 33.47 | 1.60 | 34.20 | 0.86 | 33.81 | 1.04 |
| 63.89 | 1.10 | 66.18 | 0.56 | 66.61 | 0.54 | 66.80 | 0.39 | 66.98 | 0.72 |
| 51.88 | 2.84 | 56.28 | 2.42 | 60.14 | 2.85 | 64.34 | 0.57 | 65.20 | 0.99 |

| | EMPA 114, Red wine | | | | | | | | | |
|---|---|---|---|---|---|---|---|---|---|---|
| | | | 20° C., 45 | | | | | | 20° C., 120 | |
| | 20° C., 30 min | | min. | | 20° C., 60 min | | 20° C. 90 min. | | min. | |
| | Rem. | std | Rem. | std | Rem. | std | Rem. | std | Rem. | std |
| Formulation 1 | 55.66 | 0.54 | 55.00 | 0.51 | 56.07 | 0.60 | 55.44 | 0.58 | 55.18 | 0.92 |
| Formulation 2 | 55.58 | 0.18 | 56.40 | 0.49 | 56.19 | 0.49 | 56.18 | 0.30 | 56.87 | 0.57 |
| Formulation 3 | 55.66 | 0.35 | 56.13 | 0.31 | 56.16 | 0.47 | 56.19 | 1.01 | 56.21 | 0.50 |

| 40° C., 30 min | | 40° C., 45 min. | | 40° C., 60 min | | 40° C. 90 min. | | 40° C., 120 min. | |
|---|---|---|---|---|---|---|---|---|---|
| Rem. | std | Rem. | std | Rem. | std | Rem. | std | Rem. | std |
| 56.90 | 1.11 | 57.51 | 0.63 | 55.00 | 0.87 | 55.40 | 0.65 | 54.86 | 1.57 |
| 57.74 | 0.38 | 57.40 | 0.54 | 56.50 | 0.94 | 55.52 | 0.49 | 55.87 | 1.19 |
| 58.77 | 0.40 | 57.33 | 1.26 | 56.75 | 0.31 | 56.44 | 1.23 | 56.62 | 1.34 |

| | WFK 10WB | | | | | | | | | |
|---|---|---|---|---|---|---|---|---|---|---|
| | | | 20° C., 45 | | | | | | 20° C., 120 | |
| | 20° C., 30 min | | min. | | 20° C., 60 min | | 20° C. 90 min. | | min. | |
| | Rem. | std | Rem. | std | Rem. | std | Rem. | std | Rem. | std |
| Formulation 1 | 48.93 | 0.57 | 49.50 | 0.96 | 50.63 | 0.69 | 51.18 | 1.09 | 50.90 | 0.65 |
| Formulation 2 | 49.13 | 0.90 | 50.62 | 0.65 | 50.98 | 0.75 | 51.76 | 0.95 | 53.10 | 0.43 |
| Formulation 3 | 49.23 | 0.42 | 50.37 | 0.68 | 51.02 | 0.95 | 51.95 | 0.28 | 52.40 | 0.71 |

| 40° C., 30 min | | 40° C., 45 min. | | 40° C., 60 min | | 40° C. 90 min. | | 40° C., 120 min. | |
|---|---|---|---|---|---|---|---|---|---|
| Rem. | std | Rem. | std | Rem. | std | Rem. | std | Rem. | std |
| 53.11 | 0.86 | 53.42 | 0.94 | 51.94 | 1.03 | 52.39 | 1.10 | 52.38 | 0.60 |
| 53.09 | 1.25 | 53.76 | 0.50 | 53.02 | 0.98 | 53.10 | 0.55 | 53.59 | 1.06 |
| 54.57 | 0.33 | 54.82 | 0.78 | 53.83 | 0.88 | 54.75 | 0.99 | 54.44 | 1.66 |

| | 20° C., | | 20° C., | | 20° C., | | 20° C. | | 20° C., | |
|---|---|---|---|---|---|---|---|---|---|---|
| | 30 min | | 45 min. | | 60 min | | 90 min. | | 120 min. | |
| WFK 10U | Rem. | std | Rem. | std | Rem. | std | Rem. | std | Rem. | std |
| Formulation 1 | 51.76 | 0.78 | 52.58 | 0.48 | 53.12 | 0.61 | 53.73 | 0.16 | 54.13 | 0.39 |
| Formulation 2 | 52.10 | 0.59 | 53.98 | 0.96 | 54.77 | 0.91 | 55.06 | 0.49 | 55.99 | 1.04 |
| Formulation 3 | 53.87 | 0.86 | 55.02 | 0.88 | 55.46 | 0.34 | 55.88 | 0.28 | 57.09 | 0.57 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 55.10 | 0.50 | 55.56 | 0.26 | 54.76 | 0.14 | 55.85 | 0.78 | 56.52 | 0.70 |
| | 56.07 | 1.11 | 56.30 | 0.53 | 56.92 | 0.33 | 57.25 | 0.32 | 58.30 | 0.35 |
| | 57.57 | 0.59 | 57.33 | 0.23 | 58.09 | 0.93 | 58.60 | 0.40 | 59.16 | 0.51 |

| | 20° C., | | 20° C., | | 20° C., | | 20° C. | | 20° C., | |
|---|---|---|---|---|---|---|---|---|---|---|
| EMPA 106, Carbon | 30 min | | 45 min. | | 60 min | | 90 min. | | 120 min. | |
| black/Mineral | Rem. | std | Rem. | std | Rem. | std | Rem. | std | Rem. | std |
| Formulation 1 | 34.19 | 1.42 | 36.19 | 0.75 | 35.17 | 1.86 | 36.47 | 0.26 | 38.45 | 1.57 |
| Formulation 2 | 34.87 | 4.37 | 38.64 | 1.64 | 41.07 | 0.34 | 41.71 | 0.87 | 44.76 | 1.03 |
| Formulation 3 | 37.14 | 0.28 | 36.30 | 1.62 | 36.89 | 0.99 | 37.99 | 2.86 | 38.63 | 1.83 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 36.93 | 0.69 | 38.29 | 0.95 | 37.46 | 1.64 | 40.14 | 0.96 | 40.17 | 0.75 |
| | 41.91 | 0.83 | 41.65 | 0.99 | 43.96 | 1.47 | 44.25 | 1.48 | 46.15 | 2.00 |
| | 37.39 | 1.20 | 38.89 | 0.59 | 39.30 | 1.62 | 40.68 | 1.05 | 43.19 | 0.75 |

| | 20° C., | | 20° C., | | 20° C., | | 20° C. | | 20° C., | |
|---|---|---|---|---|---|---|---|---|---|---|
| EMPA 104, Carbon | 30 min | | 45 min. | | 60 min | | 90 min. | | 120 min. | |
| black/Olive oil | Rem. | std | Rem. | std | Rem. | std | Rem. | std | Rem. | std |
| Formulation 1 | 19.25 | 1.00 | 21.68 | 0.90 | 21.29 | 0.59 | 24.15 | 1.53 | 25.19 | 0.50 |
| Formulation 2 | 24.29 | 9.57 | 22.83 | 0.66 | 24.79 | 0.54 | 26.85 | 1.20 | 27.43 | 0.63 |
| Formulation 3 | 21.36 | 1.27 | 22.35 | 0.36 | 23.97 | 0.90 | 26.38 | 1.71 | 26.67 | 1.06 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 22.20 | 0.84 | 22.87 | 1.16 | 23.12 | 1.94 | 24.94 | 0.55 | 26.62 | 1.12 |
| | 23.60 | 1.24 | 25.31 | 1.28 | 26.01 | 1.97 | 27.65 | 2.13 | 31.65 | 1.23 |
| | 23.84 | 1.25 | 25.83 | 1.26 | 27.09 | 0.61 | 28.81 | 0.84 | 31.66 | 1.11 |

| | 20° C., | | 20° C., | | 20° C., | | 20° C. | | 20° C., | |
|---|---|---|---|---|---|---|---|---|---|---|
| C-S-06, Salad | 30 min | | 45 min. | | 60 min | | 90 min. | | 120 min. | |
| dressing | Rem. | std | Rem. | std | Rem. | std | Rem. | std | Rem. | std |
| Formulation 1 | 45.16 | 1.13 | 45.83 | 0.47 | 46.34 | 0.59 | 47.29 | 0.63 | 48.06 | 0.75 |
| Formulation 2 | 56.11 | 0.77 | 59.56 | 0.76 | 61.53 | 1.75 | 63.53 | 1.05 | 65.35 | 0.90 |
| Formulation 3 | 49.68 | 1.62 | 50.34 | 0.70 | 52.09 | 2.18 | 52.85 | 1.01 | 53.90 | 1.15 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 48.74 | 0.49 | 49.19 | 1.15 | 49.54 | 0.64 | 49.79 | 0.60 | 51.93 | 0.92 |
| | 64.34 | 1.50 | 65.31 | 1.64 | 67.17 | 1.15 | 67.59 | 1.15 | 68.73 | 2.07 |
| | 54.09 | 0.74 | 55.12 | 0.38 | 55.63 | 0.84 | 56.73 | 1.17 | 57.11 | 0.94 |

| | 20° C., | | 20° C., | | 20° C., | | 20° C. | | 20° C., | |
|---|---|---|---|---|---|---|---|---|---|---|
| P-C-09, Ground | 30 min | | 45 min. | | 60 min | | 90 min. | | 120 min. | |
| nut oil | Rem. | std | Rem. | std | Rem. | std | Rem. | std | Rem. | std |
| Formulation 1 | 50.90 | 1.22 | 51.81 | 0.95 | 52.07 | 0.52 | 54.69 | 1.93 | 54.33 | 0.21 |
| Formulation 2 | 52.88 | 1.01 | 54.76 | 0.53 | 55.74 | 0.64 | 57.00 | 0.59 | 58.04 | 0.53 |
| Formulation 3 | 1.05 | 54.45 | 55.20 | 0.71 | 56.66 | 0.72 | 57.78 | 0.76 | 57.87 | 0.70 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 52.31 | 0.31 | 53.25 | 1.99 | 53.66 | 0.38 | 53.90 | 0.45 | 55.28 | 0.91 |
| | 56.84 | 1.05 | 57.50 | 1.40 | 58.86 | 0.39 | 60.20 | 0.68 | 61.63 | 0.80 |
| | 55.45 | 0.49 | 55.88 | 0.43 | 57.36 | 1.59 | 58.01 | 0.99 | 58.11 | 0.40 |

| | 20° C., | | 20° C., | | 20° C., | | 20° C. | | 20° C., | |
|---|---|---|---|---|---|---|---|---|---|---|
| | 30 min | | 45 min. | | 60 min | | 90 min. | | 120 min. | |
| 072 KC, Red Lipstick | Rem. | std | Rem. | std | Rem. | std | Rem. | std | Rem. | std |
| Formulation 1 | 19.46 | 2.35 | 25.66 | 3.01 | 29.36 | 2.07 | 41.07 | 4.46 | 52.24 | 6.65 |
| Formulation 2 | 18.85 | 0.93 | 23.54 | 3.66 | 29.33 | 4.63 | 39.38 | 3.60 | 45.03 | 2.58 |
| Formulation 3 | 24.80 | 4.07 | 32.85 | 2.74 | 45.11 | 2.38 | 51.74 | 1.66 | 63.35 | 4.24 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 59.71 | 9.83 | 72.39 | 3.33 | 78.62 | 0.96 | 81.68 | 1.06 | 80.26 | 1.44 |
| | 49.92 | 5.75 | 64.05 | 2.35 | 71.93 | 2.09 | 79.94 | 1.40 | 50.55 | 2.28 |
| | 74.71 | 3.51 | 78.95 | 3.04 | 81.06 | 1.71 | 81.86 | 1.33 | 83.05 | 0.75 |

| | 20° C., | | 20° C., | | 20° C., | | 20° C. | | 20° C., | |
|---|---|---|---|---|---|---|---|---|---|---|
| | 30 min | | 45 min. | | 60 min | | 90 min. | | 120 min. | |
| C-S-32, Sebum | Rem. | std | Rem. | std | Rem. | std | Rem. | std | Rem. | std |
| Formulation 1 | 32.08 | 0.48 | 32.90 | 1.61 | 33.52 | 1.11 | 34.49 | 0.70 | 35.65 | 0.28 |
| Formulation 2 | 35.87 | 1.55 | 37.68 | 1.77 | 38.88 | 1.37 | 40.64 | 1.62 | 39.72 | 1.84 |
| Formulation 3 | 33.19 | 0.95 | 33.28 | 0.64 | 34.21 | 0.86 | 35.65 | 0.76 | 35.96 | 1.16 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 34.72 | 1.14 | 35.05 | 0.36 | 35.80 | 0.65 | 36.55 | 0.61 | 36.60 | 1.26 |
| | 41.49 | 1.66 | 42.29 | 2.58 | 43.70 | 1.03 | 44.96 | 2.28 | 46.43 | 1.42 |
| | 35.42 | 1.45 | 37.02 | 0.86 | 36.39 | 0.72 | 37.39 | 0.68 | 37.49 | 1.11 |

| | 20° C., | | 20° C., | | 20° C., | | 20° C. | | 20° C., | |
|---|---|---|---|---|---|---|---|---|---|---|
| C-10, | 30 min | | 45 min. | | 60 min | | 90 min. | | 120 min. | |
| Milk/Nut/Pigment | Rem. | std | Rem. | std | Rem. | std | Rem. | std | Rem. | std |
| Formulation 1 | 39.69 | 0.42 | 42.12 | 0.53 | 41.00 | 0.77 | 41.61 | 1.00 | 41.42 | 1.09 |
| Formulation 2 | 52.35 | 1.05 | 56.50 | 0.47 | 57.69 | 0.53 | 60.95 | 0.58 | 61.53 | 0.66 |
| Formulation 3 | 50.50 | 2.08 | 53.37 | 0.30 | 55.66 | 1.14 | 58.48 | 1.78 | 58.82 | 0.82 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 42.43 | 0.43 | 41.83 | 0.59 | 41.44 | 0.81 | 41.56 | 0.31 | 41.56 | 0.44 |
| | 60.58 | 1.19 | 61.07 | 1.72 | 62.89 | 0.93 | 62.47 | 1.55 | 64.24 | 2.09 |
| | 59.25 | 0.88 | 59.13 | 0.75 | 60.05 | 0.97 | 61.05 | 1.19 | 62.31 | 1.45 |

| | 20° C., | | 20° C., | | 20° C., | | 20° C. | | 20° C., | |
|---|---|---|---|---|---|---|---|---|---|---|
| | 30 min | | 45 min. | | 60 min | | 90 min. | | 120 min. | |
| B-C-3, Tea | Rem. | std | Rem. | std | Rem. | std | Rem. | std | Rem. | std |
| Formulation 1 | 35.90 | 0.17 | 35.75 | 0.10 | 35.72 | 0.16 | 36.04 | 0.14 | 35.91 | 0.11 |
| Formulation 2 | 36.14 | 0.13 | 36.08 | 0.22 | 36.28 | 0.21 | 36.49 | 0.17 | 36.59 | 0.36 |
| Formulation 3 | 36.02 | 0.18 | 35.94 | 0.14 | 35.99 | 0.06 | 36.07 | 0.09 | 35.85 | 0.07 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 35.91 | 0.25 | 35.88 | 0.15 | 35.86 | 0.28 | 35.65 | 0.21 | 35.77 | 0.18 |
| | 36.42 | 0.13 | 36.41 | 0.15 | 36.27 | 0.14 | 36.31 | 0.24 | 36.38 | 0.40 |
| | 36.12 | 0.08 | 36.15 | 0.05 | 35.94 | 0.15 | 36.06 | 0.12 | 36.00 | 0.50 |

| | 20° C., | | 20° C., | | 20° C., | | 20° C. | | 20° C., | |
|---|---|---|---|---|---|---|---|---|---|---|
| | 30 min | | 45 min. | | 60 min | | 90 min. | | 120 min. | |
| B-C-2, Coffee | Rem. | std | Rem. | std | Rem. | std | Rem. | std | Rem. | std |
| Formulation 1 | 48.81 | 0.25 | 48.73 | 0.14 | 48.85 | 0.06 | 48.89 | 0.25 | 49.08 | 0.22 |
| Formulation 2 | 48.99 | 0.18 | 48.99 | 0.17 | 49.26 | 0.17 | 49.08 | 0.08 | 49.64 | 0.59 |
| Formulation 3 | 48.99 | 0.19 | 49.23 | 0.30 | 48.95 | 0.14 | 48.96 | 0.13 | 49.33 | 0.23 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 49.24 | 0.33 | 49.23 | 0.35 | 48.82 | 0.39 | 48.60 | 0.10 | 48.59 | 0.27 |
| | 49.45 | 0.23 | 49.48 | 0.12 | 49.26 | 0.24 | 48.71 | 0.30 | 49.19 | 0.19 |
| | 49.42 | 0.10 | 49.34 | 0.18 | 48.80 | 0.21 | 48.89 | 0.04 | 48.99 | 0.13 |

| | 20° C., | | 20° C., | | 20° C., | | 20° C. | | 20° C., | |
|---|---|---|---|---|---|---|---|---|---|---|
| | 30 min | | 45 min. | | 60 min | | 90 min. | | 120 min. | |
| C-S-15, Fruit Juice | Rem. | std | Rem. | std | Rem. | std | Rem. | std | Rem. | std |
| Formulation 1 | 41.32 | 0.92 | 41.26 | 0.53 | 42.31 | 0.43 | 42.58 | 0.34 | 42.72 | 0.61 |
| Formulation 2 | 41.23 | 0.61 | 42.25 | 0.34 | 43.15 | 0.86 | 43.01 | 0.36 | 44.95 | 0.32 |
| Formulation 3 | 42.09 | 0.26 | 42.49 | 0.10 | 43.18 | 0.62 | 42.86 | 0.30 | 43.62 | 0.35 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 45.17 | 0.38 | 45.24 | 0.77 | 45.11 | 1.16 | 44.31 | 1.02 | 44.08 | 1.01 |
| | 44.77 | 0.53 | 44.90 | 0.75 | 44.90 | 1.27 | 45.06 | 0.34 | 44.61 | 1.21 |
| | 46.20 | 0.30 | 45.50 | 0.78 | 44.97 | 0.37 | 45.90 | 1.01 | 45.32 | 1.32 |

| | 20° C., | | 20° C., | | 20° C., | | 20° C. | | 20° C., | |
|---|---|---|---|---|---|---|---|---|---|---|
| | 30 min | | 45 min. | | 60 min | | 90 min. | | 120 min. | |
| C-S-44, Chocolate | Rem. | std | Rem. | std | Rem. | std | Rem. | std | Rem. | std |
| Formulation 1 | 34.63 | 1.01 | 35.93 | 1.09 | 36.14 | 1.48 | 37.82 | 1.62 | 36.90 | 1.04 |
| Formulation 2 | 38.62 | 1.08 | 40.57 | 0.36 | 40.70 | 0.80 | 40.81 | 2.52 | 41.00 | 0.76 |
| Formulation 3 | 34.97 | 0.94 | 36.08 | 1.50 | 36.45 | 0.86 | 36.26 | 1.81 | 35.18 | 1.32 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 37.23 | 2.36 | 35.76 | 1.65 | 37.09 | 1.19 | 36.90 | 0.92 | 36.93 | 1.22 |
| | 40.46 | 1.47 | 40.77 | 0.79 | 41.32 | 0.74 | 40.11 | 0.77 | 41.38 | 1.83 |
| | 36.51 | 0.74 | 36.62 | 0.87 | 37.13 | 0.45 | 37.36 | 1.00 | 37.78 | 1.19 |

| | 20° C., | | 20° C., | | 20° C., | | 20° C. | | 20° C., | |
|---|---|---|---|---|---|---|---|---|---|---|
| | 30 min | | 45 min. | | 60 min | | 90 min. | | 120 min. | |
| C-S-08, Grass | Rem. | std | Rem. | std | Rem. | std | Rem. | std | Rem. | std |
| Formulation 1 | 45.91 | 0.71 | 46.84 | 0.40 | 47.38 | 0.44 | 48.67 | 0.59 | 48.78 | 0.38 |
| Formulation 2 | 47.66 | 0.96 | 49.09 | 0.34 | 50.77 | 2.04 | 50.48 | 0.35 | 50.99 | 2.11 |
| Formulation 3 | 49.50 | 0.81 | 48.60 | 3.71 | 51.28 | 0.71 | 52.56 | 0.80 | 54.00 | 1.05 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 48.85 | 0.63 | 49.23 | 0.27 | 50.37 | 0.65 | 50.38 | 0.48 | 51.91 | 1.06 |
| | 52.28 | 0.59 | 53.66 | 1.04 | 55.20 | 1.02 | 54.89 | 0.77 | 56.55 | 0.43 |
| | 54.54 | 1.31 | 55.53 | 0.83 | 56.64 | 0.27 | 58.23 | 0.96 | 58.62 | 0.73 |

| | 20° C., | | 20° C., | | 20° C., | | 20° C. | | 20° C., | |
|---|---|---|---|---|---|---|---|---|---|---|
| | 30 min | | 45 min. | | 60 min | | 90 min. | | 120 min. | |
| C-01, Motor oil | Rem. | std | Rem. | std | Rem. | std | Rem. | std | Rem. | std |
| Formulation 1 | 32.30 | 1.46 | 33.81 | 1.19 | 35.32 | 0.80 | 36.55 | 1.12 | 36.95 | 0.55 |
| Formulation 2 | 39.70 | 0.83 | 43.21 | 1.07 | 44.63 | 1.30 | 46.11 | 1.04 | 47.77 | 1.62 |
| Formulation 3 | 34.78 | 0.95 | 35.87 | 0.74 | 37.09 | 1.24 | 37.70 | 1.29 | 39.00 | 0.72 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 36.84 | 0.31 | 37.57 | 0.56 | 36.97 | 0.36 | 38.86 | 0.62 | 38.93 | 1.16 |
| | 46.96 | 2.27 | 49.56 | 1.63 | 49.28 | 1.16 | 49.79 | 0.95 | 52.40 | 1.11 |
| | 38.33 | 1.03 | 37.96 | 0.78 | 42.58 | 0.47 | 39.72 | 1.40 | 40.91 | 0.80 |

| | 20° C., | | 20° C., | | 20° C., | | 20° C. | | 20° C., | |
|---|---|---|---|---|---|---|---|---|---|---|
| | 30 min | | 45 min. | | 60 min | | 90 min. | | 120 min. | |
| C-S-17, Make-up | Rem. | std | Rem. | std | Rem. | std | Rem. | std | Rem. | std |
| Formulation 1 | 54.30 | 2.88 | 56.52 | 2.11 | 57.77 | 1.48 | 61.47 | 1.20 | 64.01 | 0.30 |
| Formulation 2 | 53.52 | 2.03 | 57.73 | 2.06 | 60.46 | 0.74 | 62.51 | 0.58 | 64.50 | 0.92 |
| Formulation 3 | 56.38 | 2.32 | 55.77 | 3.41 | 60.05 | 1.66 | 62.02 | 1.21 | 65.49 | 1.01 |
| | 40° C., | | 40° C., | | 40° C., | | 40° C. | | 40° C., | |
| | 30 min | | 45 min. | | 60 min | | 90 min. | | 120 min. | |
| | Rem. | std | Rem. | std | Rem. | std | Rem. | std | Rem. | std |
| | 61.08 | 0.46 | 61.14 | 1.25 | 61.51 | 2.07 | 66.80 | 0.86 | 68.58 | 0.93 |
| | 61.82 | 1.16 | 64.58 | 1.13 | 65.51 | 0.87 | 66.22 | 1.27 | 69.08 | 1.32 |
| | 60.69 | 1.69 | 61.29 | 2.73 | 65.62 | 1.51 | 66.72 | 1.52 | 68.02 | 0.93 |

### Example 3

### Time Dependence on Removal of Body Soils

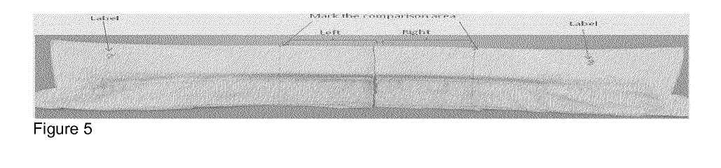

Skin-derived body soil deposition on collars, in this case cotton textile come from shirts, which have been wearing long enough to generate enough sebum/skin-derived body soil on the textile. The skin derived body soil is seen on a white textile as a gray/darker area on the textile, as seen in FIG. 5.

In order to evaluate the effect of the multi enzyme composition on the release/removal of skin-derived body soil, the collars are cut in two equal pieces in the middle of the collar. The two pieces are then marked with comparison areas in the middle part of the collar as shown in FIG. 5 and labeled with numbers. The left and right side of the collar goes to two different wash conditions.

When comparing left and right side they are always washed at the same temperature, 20° C. or 40° C., but with different washing times. The left side of the collar goes to one wash, for example the 20° C. with washing time 30 minutes and the right side of the collar goes to another wash, for example the 20° C. with wash time 45 minutes. This makes it possible to compare the washing times at a given temperature and determine the optimal washing time for removal of skin derived body soil.

In each wash a broad mix of stains, whiteness tracers and collars were washed adding up to a total load of 3 kg with cotton and cotton/polyester ballast. The formulations shown in the table 1 in example 1 were used in washing processes, all at 15° dH, in the Wascator FOM7ICLS washing machine with varying wash times from 30 and up to 120 minutes (30, 45, 60, 90 and 120 minutes) and at two different temperatures, 20° C. or 40° C. After each wash there were two rinses with water irrespectively of the washing time.

After wash, the left and right side of the collar are evaluated by an educated and trained group of people. They used the Personal Score Unit ranking (PSU), see Table 6 below, where they compared the left side collar which had been washed at one condition to the right side collar which had been washed at another condition as described above. A score of 4 in PSU reflects the best washing result, where a really strong improvement on the wash performance is seen. A scoring of minus 4 means that the left side of the washed collar is strongly worse than the right side washed collar.

TABLE 6
Ranking of collars after wash by use of personal scoring units, PSU

| Score, | |
|---|---|
| PSU | Description |
| 4 | Strongly better than control (right part very clean while the |
| | left part very dirty) |
| 3 | obviously better than left part |
| 2 | better than left part |
| 1 | slightly better than left part |
| 0 | Equal to the left part |
| −1 | Slightly worse than left part |
| −2 | worse than left part |
| −3 | obviously worse than left part |
| −4 | Strongly worse than control(left part very clean while the |
| | right part very dirty) |

When washing with a detergent comprising the multi enzyme composition it is possible to wash at the shortest time, 30 minutes, and still have the same wash performance on the collars as the wash with the longest time, 120 minutes. The same result is observed at both 20° C. and at 40° C., see table 7 and 8.

As seen in table 7 the left side collar washed at 20° C. with washing time of 30 minutes shows more or less the same wash performance (shown as PSU scoring) as the right side collar washed at 20° C. with a washing time of 120 minutes. The PSU scoring gives minus 0.05 on a scale from minus 4 to 4.

TABLE 7
Wash performance results at 20° C. defined as PSU in the
detergent containing the multi enzyme composition

| Multi enzyme | | |
|---|---|---|
| formulation at | Washing | PSU |
| 20° C. | time | scoring |
| 30 min vs | 45 min | 0.525 |
| | 60 min | 0.075 |
| | 90 min | −0.15 |
| | 120 min | −0.05 |
| 45 min vs | 60 min | −0.125 |
| | 90 min | 0.225 |
| | 120 min | 0.1625 |
| 60 min vs | 90 min | 0.25 |
| | 120 min | −0.35 |
| 90 min vs | 120 min | −0.275 |

TABLE 8
Wash performance results at 40° C. defined as PSU in the
detergent containing a multi enzyme composition

| Multi | | |
|---|---|---|
| enzyme | | |
| formulation | Washing | PSU |
| at 40° C. | time | scoring |
| 30 min vs | 45 min | −0.25 |
| | 60 min | 0.1875 |
| | 90 min | −0.025 |
| | 120 min | −0.05 |
| 45 min vs | 60 min | 0.1 |
| | 90 min | 0.1 |
| | 120 min | 0.125 |
| 60 min vs | 90 min | 0.025 |
| | 120 min | −0.1125 |
| 90 min vs | 120 min | −0.125 |

The data on which table 7 is based follow below:
Results versus 30 min.

| | | TEST- | TEST- | TEST- |
|---|---|---|---|---|
| | TESTPERSON 1 | PERSON 2 | PERSON 3 | PERSON 4 |
| collar# | | | | |
| 45 min vs | | | | |
| 30 min | | | | |
| 5 | 0 | 0 | −1 | −1 |
| 6 | 0 | 1 | 1 | 2 |
| 11 | 0 | 0 | 1 | 0 |
| 12 | 0 | 0 | 1 | 1 |
| 21 | 0 | 0 | 0 | 1 |
| 22 | 0 | −2 | −1 | −1 |
| 27 | 0 | 1 | 0 | 1 |
| 28 | 0 | −1 | 0 | −1 |
| 37 | 2 | 2 | 2 | 2 |
| 38 | 4 | 3 | 2 | 2 |
| collar# | | | | |
| 60 min vs | | | | |
| 30 min | | | | |
| 1 | 2 | 2 | 2 | 3 |
| 2 | 1 | 1 | 1 | 2 |
| 15 | 0 | 0 | −2 | −1 |
| 16 | −1 | 0 | −2 | −1 |
| 17 | −1 | −1 | 2 | 0 |
| 18 | −1 | −2 | −1 | −1 |
| 31 | 0 | 1 | 0 | 0 |
| 32 | 2 | −1 | −1 | −1 |
| 33 | 0 | 0 | 1 | 0 |
| 34 | 0 | −1 | 1 | 0 |
| collar# | | | | |
| 90 min vs | | | | |
| 30 min | | | | |
| 3 | 0 | 1 | −1 | 1 |
| 4 | 1 | 2 | 1 | 2 |
| 13 | −1 | 0 | −1 | −1 |
| 14 | −1 | −1 | −1 | −1 |
| 19 | 0 | 0 | 0 | 0 |
| 20 | −2 | −1 | −1 | −1 |
| 29 | 0 | 0 | 0 | 0 |
| 30 | 0 | 2 | 1 | 1 |
| 35 | 1 | 1 | 1 | 1 |
| 36 | −2 | −3 | −2 | −2 |
| collar# | | | | |
| 120 min | | | | |
| vs 30 min | | | | |
| 7 | 0 | 0 | 0 | 1 |
| 8 | 1 | 2 | 2 | 2 |
| 9 | −1 | 0 | −2 | −2 |
| 10 | −2 | −2 | −2 | −2 |
| 23 | −1 | 1 | −2 | 0 |
| 24 | −2 | −3 | −2 | −1 |
| 25 | 0 | 0 | 1 | −1 |
| 26 | 0 | 1 | 1 | 1 |
| 39 | 1 | 0 | 0 | 2 |
| 40 | 2 | 2 | 1 | 2 |

Results versus 45 min.

| | | TEST- | TEST- | TEST- |
|---|---|---|---|---|
| | TESTPERSON 1 | PERSON 2 | PERSON 3 | PERSON 4 |
| collar# | | | | |
| 60 min vs | | | | |
| 45 min | | | | |
| 45 | −1 | −1 | −1 | −1 |
| 46 | 0 | 1 | 2 | 1 |
| 47 | 0 | 1 | 2 | 1 |
| 48 | 0 | 1 | 1 | 0 |
| 57 | −1 | −2 | −1 | −1 |
| 58 | 0 | 0 | 0 | −1 |
| 59 | 0 | 0 | 1 | −1 |
| 60 | 0 | 2 | 0 | −1 |
| 69 | 0 | −1 | 0 | 0 |
| 70 | −1 | −1 | −1 | −2 |
| collar# | | | | |
| 90 min vs | | | | |
| 45 min | | | | |
| 43 | 0 | 1 | 1 | −1 |
| 44 | 2 | 0 | 2 | 1 |
| 49 | 0 | 2 | 2 | 0 |
| 50 | 0 | 1 | 0 | 0 |
| 55 | 0 | 0 | 0 | 0 |
| 56 | 0 | −1 | 0 | 0 |
| 61 | −1 | 0 | 1 | 0 |
| 62 | 0 | 1 | 1 | 1 |
| 67 | 0 | −1 | −1 | 1 |
| 68 | 0 | −2 | −1 | 0 |
| collar# | | | | |
| 120 min | | | | |
| vs 45 min | | | | |
| 41 | 2 | 1 | 1 | 2 |
| 42 | 2 | 2 | 1 | 1 |
| 51 | 0 | 1 | 0 | 0 |
| 52 | 0 | −1 | −1 | −1 |
| 53 | −0.5 | 0 | 0 | 0 |
| 54 | 0 | 0 | 0 | 0 |
| 63 | 0 | 0 | −1 | 0 |
| 64 | 0 | −2 | −1 | 0 |
| 65 | 0 | 2 | 1 | 0 |
| 66 | 0 | 0 | −1 | −1 |

Results versus 60 min.

| | | TEST- | TEST- | TEST- |
|---|---|---|---|---|
| | TESTPERSON 1 | PERSON 2 | PERSON 3 | PERSON 4 |
| collar# | | | | |
| 90 min vs | | | | |
| 60 min | | | | |
| 73 | 3 | 1 | 2 | −2 |
| 74 | 2 | 1 | 2 | 2 |
| 75 | 0 | −1 | −1 | −1 |
| 76 | 0 | 0 | 1 | 0 |
| 81 | 0 | 0 | 0 | 0 |
| 82 | 1 | 1 | 2 | 0 |
| 83 | −1 | 0 | −1 | −1 |
| 84 | 0 | 0 | 0 | 0 |
| 89 | 0 | −1 | 1 | 1 |
| 90 | 0 | 0 | −1 | 0 |
| collar# | | | | |
| 120 min | | | | |
| vs 60 min | | | | |
| 71 | 0 | 0 | 0 | 1 |
| 72 | 0 | 1 | −1 | −1 |
| 77 | −1 | −1 | −2 | −2 |
| 78 | 0 | −1 | 1 | 0 |
| 79 | 0 | 1 | −1 | 1 |
| 80 | −1 | −1 | −1 | 0 |
| 85 | 0 | 0 | −1 | 1 |
| 86 | −1 | −1 | −1 | 0 |
| 87 | 0 | 0 | 0 | −1 |
| 88 | 0 | −1 | −1 | 0 |

Results versus 90 min.

| collar# | | | | |
|---|---|---|---|---|
| 120 min | | TEST- | TEST- | TEST- |
| vs 90 min | TESTPERSON 1 | PERSON 2 | PERSON 3 | PERSON 4 |
| 91 | 0 | −1 | 0 | −1 |
| 92 | −1 | 0 | −1 | −1 |
| 93 | −3 | −2 | −2 | −2 |
| 94 | 0 | 0 | 0 | 1 |
| 95 | 0 | 0 | 0 | 0 |
| 96 | 0 | 0 | 0 | 0 |
| 97 | 0 | 1 | 1 | 1 |
| 98 | 0 | 0 | 0 | 1 |
| 99 | 0 | −1 | 0 | −1 |
| 100 | 0 | 0 | 0 | 0 |

The data on which table 8 is based follow below:
Results versus 30 min.

| | | TEST- | TEST- | TEST- |
|---|---|---|---|---|
| | TESTPERSON 1 | PERSON 5 | PERSON 6 | PERSON 7 |
| collar# | | | | |
| 45 min vs | | | | |
| 30 min | | | | |
| 5 | −1 | 0 | −1 | −1 |
| 6 | −1 | 0 | 0 | 0 |
| 11 | −1 | 0 | −1 | 0 |
| 12 | 0 | 0 | 1 | −1 |
| 21 | 0 | 0 | 1 | 0 |
| 22 | −2 | −1 | −1 | −1 |
| 27 | 0 | 0 | 1 | 0 |
| 28 | −1 | −1 | −1 | −1 |
| 37 | 0 | 1 | 1 | 0 |
| 38 | 0 | 1 | 0 | 0 |
| collar# | | | | |
| 60 min vs | | | | |
| 30 min | | | | |
| 1 | 0 | 0 | 0 | 1 |
| 2 | 1 | 0 | 1 | 0.5 |
| 15 | 1 | 1 | 0 | 1 |
| 16 | 0 | 1 | 1 | −1 |
| 17 | 0 | 1 | 1 | 1 |
| 18 | −1 | 0 | −1 | 0 |
| 31 | −2 | −1 | −1 | −1 |
| 32 | −1 | −1 | −1 | 0 |
| 33 | 0 | 0 | 0 | 1 |
| 34 | 2 | 2 | 1 | 1 |
| collar# | | | | |
| 90 min vs | | | | |
| 30 min | | | | |
| 3 | 1 | 1 | 1 | 1 |
| 4 | 1 | 0 | 1 | 1 |
| 13 | −1 | −1 | −1 | 1 |
| 14 | −1 | −2 | −1 | 0 |
| 19 | 1 | 0 | −1 | 0 |
| 20 | 0 | −1 | −1 | −1 |
| 29 | 0 | 0 | 1 | 0 |
| 30 | 1 | 1 | 0 | 0 |
| 35 | 0 | 0 | 0 | 0 |
| 36 | 0 | −1 | −1 | 0 |
| collar# | | | | |
| 120 min | | | | |
| vs 30 min | | | | |
| 7 | 0 | 0 | 0 | −1 |
| 8 | 1 | 1 | 1 | 1 |
| 9 | 0 | 1 | −1 | 1 |
| 10 | 0 | −1 | −1 | 0 |
| 23 | −2 | −1 | −2 | 0 |
| 24 | 0 | −2 | −2 | 0 |
| 25 | 0 | 0 | 1 | 0 |
| 26 | −1 | 0 | 1 | −1 |
| 39 | 1 | 1 | 0 | 0 |
| 40 | 0 | 2 | 1 | 0 |

Results versus 45 min.

| | | TEST- | TEST- | TEST- |
|---|---|---|---|---|
| | TESTPERSON 1 | PERSON 5 | PERSON 6 | PERSON 7 |
| collar# | | | | |
| 60 min vs | | | | |
| 45 min | | | | |
| 45 | −1 | 0 | 0 | −1 |
| 46 | 1 | 1 | 2 | 0 |
| 47 | −1 | 0 | 0 | 0 |
| 48 | −1 | 0 | −1 | −1 |
| 57 | −1 | 0 | 0 | 0 |
| 58 | −1 | 0 | 0 | 0 |
| 59 | 1 | 0 | −1 | 0 |
| 60 | 0 | 0 | −1 | 0 |
| 69 | 2 | 2 | 2 | 1 |
| 70 | 0 | 1 | 1 | 0 |
| collar# | | | | |
| 90 min vs | | | | |
| 45 min | | | | |
| 43 | −1 | 0 | 0 | 0 |
| 44 | 1 | 1 | 1 | 0 |
| 49 | −1 | 0 | 0 | 0 |
| 50 | 2 | 1 | 2 | 1 |
| 55 | 0 | 0 | 0 | 0 |
| 56 | 0 | 0 | 0 | 0 |
| 61 | −1 | −1 | −2 | 0 |
| 62 | 2 | 1 | 1 | 1 |
| 67 | −1 | −1 | −1 | −1 |
| 68 | 0 | 1 | −1 | 0 |
| collar# | | | | |
| 120 min | | | | |
| vs 45 min | | | | |
| 41 | 0 | 0 | 0 | 1 |
| 42 | 1 | −1 | 1 | 0 |
| 51 | 0 | 0 | 0 | 0 |
| 52 | 0 | 1 | 0 | 0 |
| 53 | 1 | 0 | 1 | 1 |
| 54 | 0 | 0 | −1 | 0 |
| 63 | 1 | −1 | 1 | −1 |
| 64 | −1 | −1 | −1 | −1 |
| 65 | 2 | 1 | 2 | 1 |
| 66 | 0 | −1 | −1 | 0 |

Results versus 60 min.

| | | TEST- | TEST- | TEST- |
|---|---|---|---|---|
| | TESTPERSON 1 | PERSON 5 | PERSON 6 | PERSON 7 |
| collar# | | | | |
| 90 min vs | | | | |
| 60 min | | | | |
| 73 | 0 | 0 | 1 | 0 |
| 74 | 0 | 0 | 1 | 0 |
| 75 | 1 | 0 | 0 | 0 |
| 76 | −1 | 0 | −1 | 0 |
| 81 | −1 | 1 | 0 | 1 |
| 82 | −1 | 0 | −1 | −1 |
| 83 | 1 | 0 | 1 | 0 |
| 84 | −1 | −1 | −1 | 0 |
| 89 | 0 | 0 | 1 | 0 |
| 90 | 0 | 0 | 1 | 1 |
| collar# | | | | |
| 120 min | | | | |
| vs 60 min | | | | |
| 71 | 1 | 1 | 1 | 1 |
| 72 | 1 | 1 | 1 | 0.5 |
| 77 | 1 | 0 | 0 | 0 |
| 78 | −1 | 1 | 0 | 0 |
| 79 | −2 | −1 | −1 | 0 |
| 80 | −1 | 0 | −1 | −1 |
| 85 | 0 | 1 | −1 | 0 |
| 86 | 0 | 1 | −1 | 0 |
| 87 | −1 | −1 | −1 | −1 |
| 88 | 0 | −1 | 0 | −1 |

Results versus 90 min.

| collar# | | | | |
|---|---|---|---|---|
| 120 min | | TEST- | TEST- | TEST- |
| vs 90 min | TESTPERSON 1 | PERSON 5 | PERSON 6 | PERSON 7 |
| 91 | 1 | 1 | 1 | 0 |
| 92 | −1 | 0 | −1 | 0 |
| 93 | 1 | 1 | 0 | 0 |
| 94 | 1 | −1 | 0 | 0 |
| 95 | 0 | 2 | 1 | 0 |
| 96 | −2 | 0 | −1 | −1 |
| 97 | −2 | −2 | −2 | 0 |
| 98 | 0 | 0 | 0 | 0 |
| 99 | 0 | −1 | −1 | 0 |
| 100 | −1 | 1 | 1 | 0 |

### Example 4

Reducing the washing time and the amount of surfactant.

The formulations were prepared according to table 1 below:

TABLE 1

| No. | Detergent composition | Enzyme addition |
|---|---|---|
| Formu- | Model detergent B consisting of: | No enzymes added |
| lation 1 | 7% LAS | |
| | 3% AEOS/SLES - alkyl ethoxylates/ | |
| | Sodium lauryl ether sulfate | |
| | 6.6% Non-ionic surfactant | |
| | 5.5% soap | |
| | Add up 100% with water | |
| | Surfactant content: 16.6% | |
| Formu- | Model detergent B consisting of: | Protease: 2 w/w % |
| lation 2 | 7% LAS | Liquanase 2.5 L |
| | 3% AEOS/SLES alkyl ethoxylates/ | Amylase: 0.5 w/w % |
| | Sodium lauryl ether sulfate | Stainzyme Plus 12 L |
| | 6.6% NI Non-ionic surfactant | Lipase: 0.3 w/w % Lipex |
| | 5.5% soap | 100 L |
| | Add up to 100% water | Mannanase: 0.3 w/w % |
| | Surfactant content: 16.6% | Mannaway 4 L |
| | | Pectate lyase: 0.2 w/w % |
| | | Xpect 1000 L |
| | | Cellulase: 0.3 w/w % |
| | | Celluclean 4500 L |
| Formu- | Model detergent A consisting of: | Protease: 1 w/w % |
| lation 3 | 12% LAS | Liquanase 2.5 L |
| | 5% AEOS/SLES alkyl ethoxylates/ | Amylase: 0.1 w/w % |
| | Sodium lauryl ether sulfate | Stainzyme Plus 12 L |
| | 11% Non-ionic surfactant | |
| | 5.5% soap | |
| | Add up to 100% water | |
| | Surfactant content: 28% | |

The wash liquors were used in washing processes (all at 15° dH) in the Wascator FOM7ICLS washing machine with varying wash times 30 and 120 min and at two temperatures, 20° C. or 40° C., respectively. In each wash a broad mix of in total 12 AISE and non-AISE technical stains (Double determination) were washed adding up to a total load of 3 kg with cotton and cotton/polyester ballast. After each wash there were two rinses irrespectively of the washing time. The formulations were dosed as of 50 g/wash corresponding to 3.85 g/L wash liquor. In this example, Liquanase 2.5L and Stainzyme Plus 12L is used instead of Savinase 16L and Stainzyme 12L as was used in the earlier example.

The results indicating the effect of reducing the washing time at a washing temperature of 40° C. or 20° C. at the same time maintaining the wash performance with a multi-enzyme composition containing formulation is shown in table 1. Table 1 shows the wash performance presented as remission units for the 12 stains used in this evaluation. It is shown in the table that the wash performance reach a high performance already after the 30 minutes wash when washing with the multi-enzyme composition, formulation 2, compared to formulation 3 with less amount of enzymes but with equal amount of surfactant and also versus formulation 1 without any enzymes and with full surfactant load. After 120 minutes, typically the wash performance is still high with formulation 2 and formulation 3 and formulation 1 still is increasing and is far away from the plateau. This trend, these results are shown at both 20° C. and at 40° C.

As observed from the table 1 when comparing the three formulations is the following:

There is a clear performance benefit of adding enzymes to the detergent bases tested as formulation 2 and formulation 3 have significantly higher wash performance (measured in terms of remission units at 460 nm) than formulation 1 (without enzymes)

Even with 40% reduced surfactant system the multi-enzyme formulation (formulation 2) outperforms the two other formulations in terms of wash performance as the remission units for formulation 2 is significantly higher than for formulation 1 or formulation 3 at any given wash condition.

When comparing the washing time vs. performance data it is clear that the multi-enzyme composition (formulation 2) gives the maximum performance already after around 30 minutes of washing time whereas the other formulations requires longer washing time to give their full—although notably lower—effect. This is observed as the bars for formulation 2 reaches a wash performance plateau much faster than for either of formulation 1 or 3.

TABLE 1
Raw data (remission unit numbers)

| | 20° C., | 20° C., | 40° C., | 40° C., |
|---|---|---|---|---|
| | 30 min | 120 min. | 30 min | 120 min. |
| Tomato puree | | | | |
| Formulation 1 | 59.20 | 65.33 | 63.76 | 62.99 |
| Formulation 2 | 64.71 | 71.82 | 76.51 | 79.86 |
| Formulation 3 | 59.11 | 61.50 | 65.16 | 65.46 |
| | Rem. | Rem. | Rem. | Rem. |
| Fresh Banana | | | | |
| Formulation 1 | 64.42 | 63.02 | 62.22 | 63.15 |
| Formulation 2 | 75.24 | 78.38 | 80.25 | 79.94 |
| Formulation 3 | 65.17 | 63.81 | 66.64 | 61.63 |
| Tangarine | | | | |
| Formulation 1 | 68.11 | 73.04 | 70.80 | 71.44 |
| Formulation 2 | 72.13 | 77.97 | 82.57 | 86.64 |
| Formulation 3 | 62.85 | 65.84 | 74.77 | 77.20 |
| Carrot Baby food | | | | |
| Formulation 1 | 71.45 | 75.69 | 73.94 | 77.39 |
| Formulation 2 | 78.37 | 83.57 | 84.44 | 86.06 |
| Formulation 3 | 76.97 | 80.44 | 80.73 | 80.81 |
| EMPA 101, Olive oil/carbon | | | | |
| Formulation 1 | 21.78 | 27.13 | 38.00 | 28.69 |
| Formulation 2 | 23.19 | 31.71 | 30.79 | 36.56 |
| Formulation 3 | 23.25 | 28.43 | 26.26 | 23.88 |
| EMPA 111, Blood | | | | |
| Formulation 1 | 37.34 | 37.81 | 38.63 | 42.90 |
| Formulation 2 | 51.87 | 55.88 | 59.13 | 56.69 |
| Formulation 3 | 48.98 | 52.38 | 53.72 | 49.60 |
| C-S-28, Rice starch | | | | |
| Formulation 1 | 35.61 | 38.10 | 50.30 | 40.94 |
| Formulation 2 | 54.28 | 70.86 | 67.61 | 75.07 |
| Formulation 3 | 47.17 | 69.35 | 64.51 | 71.93 |
| EMPA 106, Carbon | | | | |
| black/Mineral | | | | |
| Formulation 1 | 34.77 | 37.74 | 25.06 | 40.74 |
| Formulation 2 | 35.94 | 42.06 | 43.72 | 48.26 |
| Formulation 3 | 33.51 | 38.09 | 39.43 | 37.82 |
| EMPA 104, Carbon | | | | |
| black/Olive oil | | | | |
| Formulation 1 | 18.87 | 21.16 | 40.08 | 27.32 |
| Formulation 2 | 21.32 | 23.36 | 27.12 | 29.94 |
| Formulation 3 | 19.79 | 21.71 | 26.14 | 20.57 |
| C-S-06, Salad dressing | | | | |
| Formulation 1 | 46.99 | 47.58 | 39.95 | 51.75 |
| Formulation 2 | 60.37 | 65.93 | 68.28 | 71.06 |
| Formulation 3 | 52.53 | 54.24 | 55.47 | 52.99 |
| C-S-32, Sebum | | | | |
| Formulation 1 | 34.67 | 36.35 | 40.84 | 40.57 |
| Formulation 2 | 41.08 | 44.15 | 47.11 | 48.56 |
| Formulation 3 | 35.76 | 37.53 | 38.84 | 35.87 |
| C-01, Motor oil | | | | |
| Formulation 1 | 33.71 | 35.38 | 27.75 | 39.28 |
| Formulation 2 | 41.90 | 46.46 | 50.79 | 51.23 |
| Formulation 3 | 33.30 | 36.48 | 38.58 | 34.88 |

1-15. (canceled)

16. A composition comprising an amylase, a cellulase, a lipase, a mannanase, a pectate lyase, and a protease.

17. A detergent composition comprising a surfactant and a composition of claim 16.

18. The detergent composition of claim 17, which further comprises a hydrotrope, a builder, a co-builder, a bleaching system, a polymer, a fabric hueing agent, a mediator and/or an enzyme.

19. The detergent composition of claim 17, wherein the amount of surfactant is below 40% w/w of the detergent composition.

20. The detergent composition of claim 17, wherein the amount of surfactant is below 35% w/w of the detergent composition.

21. The detergent composition of claim 17, wherein the amount of surfactant is below 30% w/w of the detergent composition.

22. The detergent composition of claim 17, wherein the amount of surfactant is below 25% w/w of the detergent composition.

23. The detergent composition of claim 17, wherein the amount of surfactant is below 20% w/w of the detergent composition.

24. A method for cleaning a textile, comprising washing the textile in a wash liquor with a detergent composition of claim 2.

25. The method of claim 24, wherein the textile is washed for a time period that is at least 30 minutes less than a benchmark wash.

26. The method of claim 24, wherein the textile is washed for a time period that is at least 40 minutes less than a benchmark wash.

27. The method of claim 24, wherein the textile is washed for a time period that is at least 50 minutes less than a benchmark wash.

28. The method of claim 24, wherein the textile is washed for a time period that is at least 60 minutes less than a benchmark wash.

29. The method of claim 24, wherein the textile is washed for a time period that is at least 70 minutes less than a benchmark wash.

30. The method of claim 24, wherein the textile is washed for a time period that is at least 80 minutes less than a benchmark wash.

31. The method of claim 24, wherein the textile is washed for a time period that is at least 90 minutes less than a benchmark wash.


SEQUENCE LISTING (9 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 483 aa, Bacillus amyloliquefaciens
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 3 - protein, 490 aa, Bacillus sp. I633
LNNGFKKIFSITLSLLLASSILFVSGTSTANANSGFYVSGTTLYDANGNPFVMRGINHGHAWYKDQATTAIEGIANTGANTVRIVLSDGGQWTKDDIHTVRNLISLAEDNHLVAVPEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFTGGGSDGGTSPTTLYDFEGSMQGWTGSSLSGGPWAVTEWSSKGSHSLKADIQLSSNSQHYLHVIQNTSLQQNSRIQATVKHANWGSVGNGMTARLYVKTGHGYTWYSGSFVPINGSSGTTLSLDLSNVQNLSQVREIGVQFQSASDSSGQTSIYIDNVIVE

SEQ ID NO: 4 - protein, 399 aa, Bacillus subtilis [sequence omitted]

SEQ ID NO: 5 - DNA, 2322 bp, Bacillus sp.
  CDS (1)..(2322)
gcagaaggaaacactcgtgaagacaattttaaacatttattaggtaatgacaatgttaaacgcccttctgaggctggcgcattacaattacaagaagtcgatggacaaatgacattagtagatcaacatggagaaaaaattcaattacgtggaatgagtacacacggattacaatggtttcctgaratcttgaatgataacgcatacaaagctcttgctaacgattgggaatcaaatatgattcgtctagctatgtatgtcggtgaaaatggctatgcttcaaatccagagttaattaaaagcagagtcattaaaggaatagatcttgctattgaaaatgacatgtatgttattgttgattggcatgtacatgcacctggtgatcctagagatcccgtttacgctggagcagaagatttctttagagatattgcagcattatatcctaacaatccacacattatttatgagttagcgaatgagccaagtagtaacaataatggtggagctgggattccaaataatgaagaaggttggaatgcggtaaaagaatacgctgatccaattgtagaaatgttacgtgatagcgggaacgcagatgacaatatcatcattgtgggtagtccaaactggagtcagcgtcctgacttagcagctgataatccaattaatgatcaccatacaatgtatactgttcacttctacactggttcacatgctgcttcaactgagagctatccgcctgaaactcctaactctgaaagaggaaacgtaatgagtaacactcgttatgcgttagaaaacggagtagcggtatttgcgacagaatggggaacaagtcaagcaaatggagatggtggtccttattttgatgaagcagatgtatggattgagtttttaaatgaaaacaacattagttgggctaactggtctttaacgaataaaaatgaagtgtctggtgcatttacaccattcgagttaggtaagtctaacgcaaccaatcttgacccaggtccagatcatgtgtgggcaccagaagagttaagtctttcgggagaatatgtacgtgctcgtattaaaggtgtgaactatgagccaatcgaccgtacaaaatacacgaaagtactttgggactttaatgatggaacgaagcaaggatttggagtgaattcggattctccaaataaagaacttattgcagttgataatgaaaacaacactttgaaagtttcgggattagatgtaagtaacgatgtttcagatggcaacttctgggctaatgctcgtctttctgccgacggttggggaaaaagtgttgatattttaggtgctgagaagcttacaatggatgttattgttgatgaaccaacgacggtagctattgcggcgattccacaaagtagtaaaagtggatgggcaaatccagagcgtgctgttcgagtgaacgcggaagattttgttcagcaaacggacggtaagtataaagctggattaacaattacaggagaagatgctcctaacctaaaaaatatcgcttttcatgaagaagataacaatatgaacaacatcattctgttcgtgggaactgatgcagctgacgttatttacttagataacattaaagtaattggaacagaagttgaaattccagttgttcatgatccaaaaggagaagctgttcttccttctgtttttgaagacggtacacgtcaaggttgggactgggctggagagtctggtgtgaaaacagctttaacaattgaagaagcaaacggttctaacgcgttatcatgggaatttggatatccagaagtaaaacctagtgataactgggcaacagctccacgtttagatttctggaaatctgacttggttcgcggtgagaatgattatgtagcttttgatttctatctagatccagttcgtgcaacagaaggcgcaatgaatatcaatttagtattccagccacctactaacgggtattgggtacaagcaccaaaaacgtatacgattaactttgatgaattagaggaagcgaatcaagtaaatggtttatatcactatgaagtgaaaattaacgtaagagatattacaaacattcaagatgacacgttactacgtaacatgatgatcatttttgcagatgtagaaagtgactttgcagggagagtctttgtagataatgttcgttttgagggggctgctactactgagccggttgaaccagagccagttgatcctggcgaagagacgccacctgtcgatgagaaggaagcgaaaaaagaacaaaaagaagcagagaaagaagagaaagaagagtaa

SEQ ID NO: 6 - protein, 773 aa, Bacillus sp.
AEGNTREDNFKHLLGNDNVKRPSEAGALQLQEVDGQMTLVDQHGEKIQLRGMSTHGLQWFPEILNDNAYKALANDWESNMIRLAMYVGENGYASNPELIKSRVIKGIDLAIENDMYVIVDWHVHAPGDPRDPVYAGAEDFFRDIAALYPNNPHIIYELANEPSSNNNGGAGIPNNEEGWNAVKEYADPIVEMLRDSGNADDNIIIVGSPNWSQRPDLAADNPINDHHTMYTVHFYTGSHAASTESYPPETPNSERGNVMSNTRYALENGVAVFATEWGTSQANGDGGPYFDEADVWIEFLNENNISWANWSLTNKNEVSGAFTPFELGKSNATNLDPGPDHVWAPEELSLSGEYVRARIKGVNYEPIDRTKYTKVLWDFNDGTKQGFGVNSDSPNKELIAVDNENNTLKVSGLDVSNDVSDGNFWANARLSADGWGKSVDILGAEKLTMDVIVDEPTTVAIAAIPQSSKSGWANPERAVRVNAEDFVQQTDGKYKAGLTITGEDAPNLKNIAFHEEDNNMNNIILFVGTDAADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVAFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTEPVEPEPVDPGEETPPVDEKEAKKEQKEAEKEEKEE

SEQ ID NO: 7 - protein, 773 aa, Bacillus sp.; identical to SEQ ID NO: 6

SEQ ID NO: 8 - protein, 269 aa, Bacillus clausii
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISAPASYANAMAVGATDQNNNRASFSQYGPGLDIVAPGVNVQSTYPGSTYASLNGTSMATPHVAGAAALVKQKNPSWSNVQIRNHLKNTATSLGSTNLYGSGLVNAEAATR

SEQ ID NO: 9 - protein, 483 aa, Bacillus sp.
HHNGTNGTLMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVKAVEVNPNNRNQEVSGEYTIEAWTKFDFPGRANTHSNFKWRWYHFDGVDWDQSRKLNNRIYKFRTKAWDWEVDTEFGNYDYLLYADIDMDHPEVVNELRNWGVWYTNTLGLDGFRIDAVKHIKYSFTRDWINHVRSAIGKNMFAVAEFWKNDLGAIENYLNKTNWNHSVFDVPLHFNLYYASKSGGNYDMRQIFNGTVVQKHPTHAVTFVDNHDSQPEESLESFVREWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNKAGTVTINADGWGNFSVNGGSVSIWVNK